(19) United States
(12) Patent Application Publication
(10) Pub. No.: US 2007/0053855 A1
(43) Pub. Date: Mar. 8, 2007

(54) Personal care products incorporating cellulosic fatty acid esters

(76) Inventors: Michael Charles Shelton, Kingsport, TN (US); Eric Eugene Ellery, Kingsport, TN (US); Chung-Ming Kuo, Kingsport, TN (US); Debra Tindall, Kingsport, TN (US); Terry Ann Oldfield, Kingsport, TN (US); Jason Alan Smith, Kingsport, TN (US)

Correspondence Address: William K. McGreevey;Eastman Chemical Company, P.O. Box 511, Kingsport, TN, 37662-5075, US

(21) Appl. No.: 11/521,247

(22) Filed: Sep. 14, 2006

Related U.S. Application Data
(63) Continuation-in-part of application No. 11/226,802, filed on Sep. 14, 2005.
(60) Provisional application No. 60/610,367, filed on Sep. 16, 2004.
(60) Provisional application No. 60/782,784, filed on Mar. 16, 2006.

(51) Int. Cl. A61K 8/73

(52) U.S. Cl. 424/59; 424/63; 424/64; 424/70.13; 424/70.7; 424/73

(57) ABSTRACT

Cellulose ester compounds and compositions that include such cellulose ester compounds dissolved in lipophilic solvents.

### CROSS-REFERENCE TO RELATED APPLICATIONS

This application is a continuation-in-part of U.S. patent application Ser. No. 11/226,802 filed on Sep. 14, 2005, which claims the benefit of U.S. Provisional Patent Application No. 60/610,367 filed Sep. 16, 2004. This application further claims priority to U.S. Provisional Patent Application No. 60/782,784 filed on Mar. 16, 2006. The disclosures of each of the foregoing applications are incorporated herein by reference in their entirety.

### FIELD OF THE INVENTION

The present invention relates to cellulose ester compounds and compositions that include such cellulose ester compounds. More particularly, the present invention relates to compositions containing cellulose ester compounds with the ability to dissolve in lipophilic solvents.

### BACKGROUND OF THE INVENTION

Cellulose ester compounds are cellulose molecules having ester moieties substituted for one or more hydroxyls pendant to the 1,4′-O-β-bonded anhydroglucose monomer units linked in series to form the cellulose polymeric backbone. They can be used, for example, as a film forming component or thickener in compositions to give the compositions desired physical properties. Cosmetics and personal care products that are oil-based or have an oil phase have limited durability on the lips or skin. For example, color cosmetics wear off after a limited amount of time when subjected to forces of smudging or smearing, especially when accompanied by perspiration. Skin care products, in the case of sunscreens for example, rub off when contacted by clothing or rinse off while swimming. To improve durability and water resistance of such compositions, it is desirable that they contain an oil-soluble film-former. Compositions such as color cosmetics, deodorants, skin care creams and lotions, and hair preparations need to be thickened so that they can be applied in the form of a stick or can be poured into and contained in the hand and applied with the fingers. Thickening is also beneficial so that compositions stay where they are placed rather than running or dripping away from the intended substrate. It is desirable for a thickened composition to be shear thinning to provide ease of spreading or when sprayed to provide a fine droplets and even distribution. Accordingly, there is a need for a personal care item that has good performance, long lasting, suitable texture and is easy to apply. There is also a need for cellulose ester compounds that can be dissolved in oil-based cosmetic and personal care compositions at concentrations adequate to confer favorable physical properties such as thickening and film formation.

### SUMMARY OF THE INVENTION

The problems in the art are solved by providing cellulose ester compounds that dissolve in lipophilic solvents at higher concentrations than previously achievable. In some embodiments, the concentrations are higher than 10%, 20%, or even 50% by weight at room temperature and standard pressure. These higher concentrations allow preparation of cosmetic and personal care compositions that have desirable physical properties such as thickening, and film-formation. Improved film-formation in some personal care products can improve the product's function as a carrier for other components as well as product properties such as smudge resistance, transfer resistance; durability, water resistance, moisture barrier properties, and carrier for additives, actives, and/or pigments.

The invention further provides compositions containing one or more cellulose ester compounds of the present invention along with one or more lipophilic solvent, and uses of such compositions in personal care and cosmetic applications.

The invention further provides personal care products containing the compositions of the present invention.

### DETAILED DESCRIPTION OF THE INVENTION

Definitions

As used throughout this application, “cellulose” shall mean any cellulose from any natural or artificial source. Chemically, cellulose is described, for example, as a polymer containing a repeating series of anhydroglucose monomer units that are covalently bonded in series by 1,4′-O-β-glycosidic linkages. Examples of useful cellulose sources include hardwood pulp, softwood pulp, cotton linters, bacterial cellulose, and regenerated cellulose. Source materials that include other compounds (e.g. hemicellulose) may also be used.

As used throughout this application, the term “cellulose compound” shall include cellulose as well as any compound based upon cellulose having substitution of one or more of the hydroxyl groups present on naturally occurring cellulose with another moiety. The substitution can be with any moiety (e.g. an ester, an ether, thioester, thioether (sulfide), hydroxyl, phosphate, carbonate, amine, imine, amide, nitrate, nitrile, urethane (carbamate ester), urea, thiourea, silane, sulfate, sulfonic acid, azole, phenol, pyridine or pyrimidine moieties or linkages, or any combination of the foregoing).

As used throughout this application, the term “cellulose ester compound” shall mean any cellulose compound that possesses one or more ester moieties substituted for one or more of the free hydroxyl moieties that occur on naturally occurring cellulose. The substitution is such that the oxygen in the hydroxyl moiety is replaced with the (single bonded) oxygen of the ester moiety. Any compound fitting this description falls within this definition. Thus, cellulose ester compounds may be the reaction product of esterification of unmodified cellulose, esterification of a cellulose compound that is already modified (including cellulose compounds that are themselves cellulose esters, such as, cellulose acetate, cellulose propionate, cellulose butyrate, cellulose acetate propionate, cellulose acetate butyrate, carboxymethylcellulose, carboxymethylcellulose acetate butyrate etc.), or esterification of a combination of modified and unmodified cellulose. As a result, the cellulose ester compounds may have two or more types of substitutions or derivitizations, including two or more types that occur on some of the same monomers. Furthermore, cellulose ester compounds may be the reaction product of esterification with two or more different types of ester moieties, such as the reaction product of an esterification reaction using a mixture of carboxylic acids, acid chlorides, or both. As used throughout this application, the reference to a molecule as the “reaction product” of specified reactants is provided as a convenient way to describe the structure of the molecule, and not as a limitation to molecules made by specific methods or using specific reactants. Thus, any molecule having the molecular structure described by reference to a reaction product, but obtained by other methods or from other reactants, will be within the meaning of “reaction product” as that term is used throughout this application. The timing, order, and nature of esterification reactions, as well as whether such reactions occur simultaneously, are not critical because any cellulose compound that has one or more type of ester moiety is within the scope of this definition. Some examples of processes for esterifying cellulose compounds are: acid-catalyzed transesterification using fatty acids; base-catalyzed transesterification using fatty acids; acid-catalyzed direct esterification using fatty acid anhydrides; acid-catalyzed direct esterification using fatty acid chlorides; and acid-catalyzed direct esterification using fatty acid mixed anhydrides. Processes and procedures used to prepare the cellulose ester compounds are described in greater detail in Gedon, S.; Fengl, R. “Cellulose Esters,” Kirk-Othmer Encyclopedia of Chemical Technology, 4th Ed., vol. 5, John Wiley & Sons, New York, 1993, pp. 496-529.

Examples of cellulose ester compounds include cellulose hexanoate, cellulose nonanoate, cellulose laurate, cellulose palmitate, cellulose stearate, cellulose behenate, cellulose 2-ethylbutyrate, cellulose 2-ethylhexanoate, cellulose iso-butyrate, cellulose iso-valerate (cellulose iso-pentanoate), cellulose iso-nonanoate, cellulose iso-decanoate, cellulose iso-stearate, cellulose pivalate (cellulose neo-pentanoate), cellulose neo-heptanoate, cellulose neo-decanoate, cellulose neo-undecanoate, cellulose neo-dodecanoate, cellulose neo-stearate, cellulose oleate (cellulose octadecenoate), cellulose linoleate (cellulose octadecadienoate), cellulose linolenate (cellulose octadecatrienoate); cellulose acetate hexanoate, cellulose acetate nonanoate, cellulose acetate laurate, cellulose acetate palmitate, cellulose acetate stearate, cellulose acetate behenate, cellulose acetate 2-ethylbutyrate, cellulose acetate 2-ethylhexanoate, cellulose acetate iso-butyrate, cellulose acetate iso-valerate (cellulose acetate iso-pentanoate), cellulose acetate iso-nonanoate, cellulose acetate iso-decanoate, cellulose acetate iso-stearate, cellulose acetate pivalate (cellulose acetate neo-pentanoate), cellulose acetate neo-heptanoate, cellulose acetate neo-decanoate, cellulose acetate neo-undecoanoate, cellulose acetate neo-dodecanoate, cellulose acetate neo-stearate, cellulose acetate oleate (cellulose acetate octadecenoate), cellulose acetate linoleate (cellulose acetate octadecadienoate), cellulose acetate linolenate (cellulose acetate octadecatrienoate), cellulose propionate hexanoate, cellulose propionate nonanoate, cellulose propionate laurate, cellulose propionate palmitate, cellulose propionate stearate, cellulose propionate behenate, cellulose propionate 2-ethylbutyrate, cellulose propionate 2-ethylhexanoate, cellulose propionate iso-butyrate, cellulose propionate iso-valerate (cellulose propionate iso-pentanoate), cellulose propionate iso-nonanoate, cellulose propionate iso-decanoate, cellulose propionate iso-stearate, cellulose propionate pivalate (cellulose propionate neo-pentanoate), cellulose propionate neo-heptanoate, cellulose propionate neo-decanoate, cellulose propionate neo-undecoanoate, cellulose propionate neo-dodecanoate, cellulose propionate neo-stearate, cellulose propionate oleate (cellulose propionate octadecenoate), cellulose propionate linoleate (cellulose propionate octadecadienoate), cellulose propionate linolenate (cellulose propionate octadecatrienoate); cellulose butyrate hexanoate, cellulose butyrate nonanoate, cellulose butyrate laurate, cellulose butyrate palmitate, cellulose butyrate stearate, cellulose butyrate behenate, cellulose butyrate 2-ethylbutyrate, cellulose butyrate 2-ethylhexanoate, cellulose butyrate iso-butyrate, cellulose butyrate iso-valerate (cellulose butyrate iso-pentanoate), cellulose butyrate iso-nonanoate, cellulose butyrate iso-decanoate, cellulose butyrate iso-stearate, cellulose butyrate pivalate (cellulose butyrate neo-pentanoate), cellulose butyrate neo-heptanoate, cellulose butyrate neo-decanoate, cellulose butyrate neo-undecoanoate, cellulose butyrate neo-dodecanoate, cellulose butyrate neo-stearate, cellulose butyrate oleate (cellulose butyrate octadecenoate), cellulose butyrate linoleate (cellulose butyrate octadecadienoate), cellulose butyrate linolenate (cellulose butyrate octadecatrienoate); cellulose acetate propionate hexanoate, cellulose acetate propionate nonanoate, cellulose acetate propionate laurate, cellulose acetate propionate palmitate, cellulose acetate propionate stearate, cellulose acetate propionate behenate, cellulose acetate propionate 2-ethylbutyrate, cellulose acetate propionate 2-ethylhexanoate, cellulose acetate propionate iso-butyrate, cellulose acetate propionate iso-valerate (cellulose acetate propionate iso-pentanoate), cellulose acetate propionate iso-nonanoate, cellulose acetate propionate iso-decanoate, cellulose acetate propionate iso-stearate, cellulose acetate propionate pivalate (cellulose acetate propionate neo-pentanoate), cellulose acetate propionate neo-heptanoate, cellulose acetate propionate neo-decanoate, cellulose acetate propionate neo-undecoanoate, cellulose acetate propionate neo-dodecanoate, cellulose acetate propionate neo-stearate, cellulose acetate propionate oleate (cellulose acetate propionate octadecenoate), cellulose acetate propionate linoleate (cellulose acetate propionate octadecadienoate), cellulose acetate propionate linolenate (cellulose acetate propionate octadecatrienoate); cellulose acetate butyrate hexanoate, cellulose acetate butyrate nonanoate, cellulose acetate butyrate laurate, cellulose acetate butyrate palmitate, cellulose acetate butyrate stearate, cellulose acetate butyrate behenate, cellulose acetate butyrate 2-ethylbutyrate, cellulose acetate butyrate 2-ethylhexanoate, cellulose acetate butyrate iso-butyrate, cellulose acetate butyrate iso-valerate (cellulose acetate butyrate iso-pentanoate), cellulose acetate butyrate iso-nonanoate, cellulose acetate butyrate iso-decanoate, cellulose acetate butyrate iso-stearate, cellulose acetate butyrate pivalate (cellulose acetate butyrate neo-pentanoate), cellulose acetate butyrate neo-heptanoate, cellulose acetate butyrate neo-decanoate, cellulose acetate butyrate neo-undecoanoate, cellulose acetate butyrate neo-dodecanoate, cellulose acetate butyrate neo-stearate, cellulose acetate butyrate oleate (cellulose acetate butyrate octadecenoate), cellulose acetate butyrate linoleate (cellulose acetate butyrate octadecadienoate), cellulose acetate butyrate linolenate (cellulose acetate butyrate octadecatrienoate) and the like. Cellulose ester compounds that are derivatized with other moieties in addition the ester moiety (for example, carboxymethylcellulose and carboxymethylcellulose acetate butyrate, cellulose acetate phthalate, hydroxypropyl cellulose and the like) are also included.

As used throughout this application, the term “ester group” or “ester moiety,” as used to describe a group substituted for free hydroxyl groups on cellulose compounds shall include any group or moiety having the structure —O(C═O)—R in which the oxygen that is not double bonded to carbon is bonded to the one of the carbons of an anhydroglucose residue of the cellulose compound. R may by any type of organic group including saturated or unsaturated, branched or straight chain alkyl groups or cyclic groups, or combinations thereof, of any size. R may further include aromatic groups and any other type of substitution or other structure that includes atoms other than carbons and hydrogens. Common examples of such atoms include nitrogen, oxygen, aluminum, silicon and sulfur. Thus, R may include, by way of example only, one or more ester, ether, thioester, thioether (sulfide), hydroxyl, phosphate, carbonate, amine, imine, amide, nitrate, nitrile, urethane (carbamate ester), urea, thiourea, silane, sulfate, sulfonic acid, azole, phenol, pyridine or pyrimidine moieties or linkages, or any combination of the foregoing.

As used throughout this application, the term “lipophilic solvents” means solvents having solubility in water that is in the “sparingly soluble” range, or lower. Persons of ordinary skill in the art will understand that, for compounds that are “sparingly soluble in water,” the quantity of water needed to dissolve one gram of the compound will be in the range beginning at about 30 mL and ending at about 100 mL. Compounds having solubility lower than “sparingly soluble” (i.e. “slightly soluble,” “very slightly soluble,” or “practically insoluble”) in water will require greater volumes of water to dissolve the compounds.

Examples of suitable lipophilic solvents include hydrocarbons, alkyl esters, fats and oils, fatty acids, fatty alcohols, and silicone oils. Examples of hydrocarbons include isoparaffins, hydrogenated poly(iso-butene), isododecane, isoeicosane, isohexadecane, isopentane, microcrystalline wax, mineral oil, mineral spirits, paraffin, petrolatum, squalene, polyethylene, natural waxes such as carnauba wax and candelilla wax, hydrocarbons set forth on pages 2136 and 2137 of the CTFA International Cosmetic Ingredient Handbook, Tenth Edition, 2004, and mixtures of two or more of any of the foregoing. Examples of suitable alkyl esters are those in which the inventive cellulose ester is soluble, preferably where the alkyl portion has at least eight carbon atoms. These include alkyl acetates, alkyl behenates, alkyl lactates, alkyl benzoates, alkyl salicylates, typical alkyl fatty acid esters such as alkyl stearates, alkyl palmitates, alkyl myristates, and alkyl laurates, and mixtures thereof. Examples of fats and oils, further defined as glyceryl esters of fatty acids (triglycerides), also include synthetically prepared esters of glycerin and fatty acids. Examples include soybean oil, corn oil, canola oil, olive oil, sunflower oil, triolein, tristearin, caprylic/capric triglyceride, and mixtures thereof. Examples of fatty acids include valeric acid, heptylic acid, caprylic acid, lauric acid, myristic acid, and palmitic acid, behenic acid, capric acid, caproic acid, coconut acid, oleic acid, linoleic acid, palmitic acid, isopalmitic acid, stearic acid, isostearic acid, and mixtures thereof. Examples of suitable fatty alcohols include C₉-C₃₀ alcohols, branched and straight chain. These include, for example, lauryl alcohol, isolauryl alcohol, cetyl alcohol, isocetyl alcohol, stearyl alcohol, isostearyl alcohol, octyldodecanol, octyl tetradecanol, dodecyl hexadecanol, hexadecyl eicosanol, and mixtures thereof. Examples of suitable silicone oils include those compatible with an oil-based solution of the cellulose ester, including volatile and non-volatile silicone oils, linear and cyclic. Examples include dimethicone, hexadecyl methicone, stearyl dimethicone, cyclomethicone, cyclopentasiloxane, phenyl trimethicone, and mixtures thereof.

As used throughout this application, “reference solvent” or “reference lipophilic solvent” shall mean isododecane, CAS No. 31807-55-3.

As used throughout this application, the terms “degree of substitution”, “DS” and “DS/AGU” each refer to the average number of free hydroxyl groups substituted by ester moieties per anhydroglucose ring of the cellulose compound. The theoretical maximum DS is 3, since that is the number of free hydroxyl groups per anhydroglucose ring on unsubstituted cellulose. DS can be determined by proton NMR in dimethylsulfoxide-d₆ [(CD₃)₂SO; DMSO-d₆], tetrahydrofuran-d₈ [C₄D₈O; THF-d₈], chloroform-d [CDCl₃] or tetrachloroethane-d (TCE-d) (or an equivalent solvent) containing several drops of trifluoroacetic acid [TFA]. For example, using proton NMR, DS can be determined by ¹H NMR in dimethylsulfoxide-d₆ [(CD₃)₂SO; DMSO-d₆] or tetrahydrofuran-d₈ [C₄D₈O; THF-d₈] containing several drops of trifluoroacetic acid to shift any free hydroxylprotons downfield. “Degree of Substitution” and its synonyms can be used to describe the number of substitutions with a particular single moiety (e.g. the DS for neoheptanoate), the number of substitutions with a group of moieties (e.g. the DS for all esters with branched saturated alkyl chains) or the total number of substitutions on the cellulose ester compound (i.e. the “total DS”).

When determining the total DS of an ester, procedures are altered as necessary to minimize the possibility that NMR results do not include moles of residual reactants as part of the moles of ester. For example, where esterification is performed with carboxylic acids, procedures are corrected to allow subtraction of moles of free acid from the value for moles of total acid, which includes both free acid and ester values. In this procedure a sample of the cellulose ester compound is dried to be essentially free of solvent and water. 20 milligrams of the cellulose ester compound is dissolved in about 1 milliliter of TCE-d and heated to as high as 80° C. with stirring if necessary to dissolve the cellulose ester. The solution is then allowed to cool to room temperature and at least 0.1 ml of trichloroacetyl isocyanate is added to the solution, followed by stirring for at least 5 minutes. Samples are then subjected to proton NMR spectrum (at 80° C.) within a few hours of preparation using the normal proton NMR parameters, but with 64 scans. The resulting NMR data provides a peak for free acid present as well as a peak for total acid (acids and esters combined). Both peaks are integrated and the peak for free acid is subtracted from the total acid peak to provide a value for total esterified acid. This value is used to calculate DS. With this method, an internal check is available by comparing the moles for reacted hydroxyls of the cellulose (using the hydroxyl peak) since the derivatizing agent reacts with both the unesterified hydroxyls of the cellulose and the unesterified acid. The moles of hydroxyl is calculated and divided by the moles of backbone to yield a value for number of remaining hydroxyl groups per anhydroglucose. The value 3.00 minus this value hydroxyl should yield the sane DS value for esterified acid as calculated using the other method.

As used throughout this application, the terms “degree of polymerization” and “DP” and “DPᵥ” shall each mean the number of monomers in a given polymer chain as calculated based on Intrinsic viscosity (IV) using the following method. IV is first determined using a RPV-1 Polymer Viscometer, available from Rheotek USA Inc., LaGrange, Ky., or any equivalent device. The Rheotek instrument requires sample moisture content to perform its calculations. Moisture testing on cellulose ester compound is conducted by comparing weight before and after drying. Drying is performed by placing 4.0 to 5.0 gram of a sample of the compound in a 105 degrees C. oven for at least 4 hrs to dry. The sample is allowed to cool for at least 1 hour in a dessicator before reweighing. Based on the change in weight, percent moisture in the sample is calculated. To calculate IV, approximately 0.25 grams sample is placed into a container with 50 mL of water and stirred magnetically for 1 to 5 minutes as needed to disperse and wet the sample with water. 50 mL of cupriethylenediamine hydroxide (Cuene) is then dispensed into the container under a nitrogen purge and allow to stir for at least one minute, then sample is immediately capped and stirred until the sample is completely dissolved. The sample is tested by Rheotek Viscometer to determine the flow time using a 25.0 degrees C. constant temperature mineral oil bath. The average flow time, based on two closest replicates, is used to calculate the IV by the software provided by Rheotek. The Rheotek instrument tests each sample for average flow time and related software calculates the IV on a personal computer. Accurate methods that are equivalent to the foregoing may also be used.

Once IV is known, DPᵥ or degree of polymerization values are calculated by the following formula:

$$\overline{{DP}_{v}} = ( 1.65 ) ( {[ η ]}^{\frac{1}{0.9}} )$$

where η is the measured IV that is reported in units of deciliters per gram by the Rheotek instrument but converted to milliliters per gram in order to satisfy the given equation.

Unless specifically indicated otherwise, as used throughout this application the word “include” and other forms of this word (e.g. “including”) where used to describe a list or description of category of things shall mean that the list or description is not intended to provide examples and is not intended to be limiting or exclusive.

As used throughout this application, “room temperature and standard pressure” shall mean a temperature of 25 degrees Celsius and a pressure of one atmosphere.

Cellulose Ester Compounds

The cellulose ester compounds of the present invention are useful because of their ability to be dissolved in lipophilic solvents at advantageously high concentrations. This property is described herein by stating whether the cellulose ester compound dissolves in the isododecane reference solvent at specified concentrations by weight at room temperature and standard pressure. Thus, in some embodiments the cellulose ester compound dissolves in isododecane at concentrations of at least about 5% by weight at room temperature and standard pressure. In some embodiments, the cellulose ester compound dissolves in isododecane at concentrations of at least about 10% by weight at room temperature and standard pressure. In some embodiments, the cellulose ester compound dissolves in isododecane at concentrations of at least about 15% by weight at room temperature and standard pressure. In some embodiments the cellulose ester compound dissolves in isododecane at concentrations of at least about 20% by weight at room temperature and standard pressure, and so on. Thus, embodiments can be described in which the cellulose ester compound dissolves in isododecane at room temperature and standard pressure at higher weight concentrations such as of at least about 25%, at least about 30%, at least about 35%, at least about 40%, at least about 45%, at least about 50% by weight. Throughout this application, “concentration by weight” is defined as the weight of the cellulose ester compound divided by the total weight of the entire sample (compound, solvent, and any other components), multiplied by 100%.

It has been discovered that cellulose ester compounds having excellent solubility properties may be obtained using cellulose having a total DS of about 2.5 or higher for substitution of hydroxyl groups with one or more ester moieties having at least about 6 carbon atoms (including the carbon that is part of the ester group). In some embodiments, the ester groups have at least about 7 carbon atoms, or at least about 8 carbon atoms. The carboxylic acid esters on the cellulose compound may have all the same structure or two or more different structures.

The invention, however, is not limited to specific DS ranges. In some embodiments, the cellulose ester compounds have a total DS in the range of 0.5 to 3.0. An optimal DS or minimum DS that achieves a desired solubility can readily be determined by routine testing in which the cellulose ester compound is simply manufactured with varying DS values and the solubility of each DS value in the reference solvent is tested. Thus, the cellulose ester compound may be described as having a DS value above a specified minimum, for example, at least about any of the following: 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, or 2.9. Alternatively, the cellulose ester compound may be described as having a DS value within a specified range, for example, at least about any of the following: 0.5-0.6, 0.6-0.7, 0.7-0.8, 0.8-0.9, 0.9-1.0, 1.0-1.1, 1.1-1.2, 1.2-1.3, 1.3-1.4, 1.41-1.5, 1.5-1.6, 1.6-1.7, 1.7-1.8, 1.8-1.9, 1.9-2.0, 2.0-2.1, 2.1-2.2, 2.2-2.3, 2.3-2.4, 2.4-2.5, 2.5-2.6, 2.6-2.7, 2.7-2.8, 2.8-2.9, or 2.9-3.0. Broader ranges combining any of the foregoing ranges may also be used, for example, 0.5-1.0, 1.0-1.5, 1.0-2.0, 1.5-2.0, 2.0-3.0 2.0-2.5 or 2.5-3.0, or ranges such as 1.5-1.8, 1.6-1.8, 1.7-2.0, 1.8-2.2, 2.0-2.2, 2.2-2.6, 2.3-2.5, 2.4-2.6, 2.5-2.7, 2.4-2.8 and the like. Each of the above DS values may be the DS for a given moiety or group of moieties, or the total DS for the cellulose ester compound.

It has further been discovered that the above cellulose ester compounds have especially advantageous solubility in lipophilic solvents if the ester moieties on such compounds are branched and saturated, unbranched and saturated, or branched and unsaturated. Examples of branched ester moieties include: esters having a tert-butyl terminus (also may be referred to as a quaternary carbon terminus) on any chain (primary or side); esters having a saturated iso-alkyl group, either with or without further branching; and esters having an alkyl structure that is branched at the alpha carbon (that is the carbon that is directly bonded to the carbon in the carboxylic acid group). Some examples of ester moieties having tert-butyl groups include neo-heptanoate, neo-decanoate, neo-undecanoate, neo-dodecanoate, and neo-stearate. Cellulose ester compounds that have mixed ester moieties that are the residues of esterification with mixtures of neo-acids are also within the scope of the invention. Examples of such mixtures containing neo-acids include Neo 910 Acid ExxonMobil Chemical, Neo 913 (ExxonMobil Chemical), and commercially available blends of C9-C28 neo-based derivatives. Some examples of esters having saturated iso-alkyl groups include iso-octanoate, iso-decanoate, 3,5,5-trimethyl-hexanoate, and iso-stearate. Some examples of esters having an alkyl structure that is branched at the alpha carbon include 2-methylpentanoate (2-methylvalerate), 2-methylhexanoate, 2-methyloctanoate, 2-methyldecanoate, 2-methylstearate, 2-ethylbutyrate, 2-ethylpentanoate (2-ethylvalerate), 2-ethylhexanoate, 2-ethyloctanoate, 2-ethyldecanoate, 2-ethylstearate, 2,2-dimethylbutyrate, 2-ethyl-2-methylbutyrate, 2,2-diethylbutyrate, 2,2-dimethylvalerate (2,2-dimethylpentanoate), 2-ethyl-2-methylvalerate (2-ethyl-2-methylpentanoate), 2,2-diethylvalerate (2,2-diethylpentanoate), 2,2-dimethylhexanoate, 2,2-diethylhexanoate, 2,2-dimethyloctanoate, 2-ethyl-2,5-dimethylhexanoate, 2,2-dimethyl-3-hydroxypropanoate. Cellulose ester compounds that have mixed ester moieties that are the reaction products of esterification with mixtures of branched acids are also within the invention. Some examples of ester moieties having an unsaturated alkyl group include myristoleate (9-tetradecenoate), palmitoleate (9-hexadecenoate), oleate (cis-9-ocatdecenoate), elaidate (trans-9-ocatdecenoate), erucate (cis-13-docosenoate), linoleate (9,12-octadecenoate), linolenate (9,12,15-octadecatrienoate), and the like. Cellulose ester compounds that have mixed ester moieties that are the residues of esterification with mixtures of unsaturated acids are also within the invention.

The cellulose ester compound may also be the product of an esterification reaction involving a mixture of compounds, resulting in a cellulose ester compound having different types of ester moieties. This is used in several embodiments prepared from esterification with fatty acids because many commercial fatty acid products are derived from natural sources and therefore contain a mix of different acids and potentially other compounds. For example, nonanoic acid, also referred to as pelargonic acid, is a colorless to yellowish oil and may be derived, for example, by the oxidation of nonyl alcohol, nonyl aldehyde, or oleic acid. Thus, cellulose ester compounds possessing nonanoate ester moieties may be produced, for example, by esterification using a commercial product that contains 96% nonanoic acid product and 4% 2-methyloctanoic (e.g. Aldrich; Catalog # N29902), possibly resulting in a cellulose ester product having two moieties. Similarly, many products sold as iso-stearic acid products used in the synthesis of several derivatives are actually commercial sources that contain a mixture of isomeric C18 fatty acids (e.g. A& E Connock, Perfumery and Cosmetics, LTD).

It will be understood that invention relates to the structure of the cellulose ester compound and that neither the process nor the reactants used to prepare the esters are critical to the invention. Thus, references, for example, to the ester moiety as residues or reaction products of carboxylic acid esterification reactions are used only as a convenient method for describing the structures and do not limit the inventions to compounds actually prepared by reactions involving carboxylic acids. Any compounds matching the structural descriptions are within the invention, regardless of whether produced by (for example) reactions with carboxylic acids, reactions with acid chlorides, reactions with anhydrides, or any other means.

In some embodiments, the DPᵥ of cellulose ester compounds is of a range that is advantageous to solubility in lipophilic solvents. In some embodiments, the DPᵥ is less than about 4000. In some embodiments, the DPᵥ is less than about 3500. Embodiments also exist in which the DPᵥ is less than about 3000, less than about 2500, less than about 2000, less than about 1500, less than about 1000, or less than about 500. As with DS, the ideal range in such embodiments can be readily determined by routine experimentation. DP will of course determine molecular weight. Thus, in some embodiments, the cellulose ester compounds of the invention typically have a weight average molecular weight (M_{w}) as measured by gel permeation chromatography in THF against a polystyrene standard of about 20,000 to about 8,000,000 Daltons against a polystyrene standard.

In some embodiments, the cellulose compounds of the present invention are mixed ester compounds having other ester groups thereon in addition to the branched or unsaturated groups. Examples of the other ester groups include acetate, propionate, the butyrates, and the pentanoates. In some embodiments involving mixed esters, high solubility can be attained even though the DS for the branched and/or unsaturated ester moieties is less than 2.5, for example, at least about 0.8, at least about 1.0, at least about 1.5, or at least about 2.0.

It has also been found that in some embodiments with cellulose ester compounds that include both acetate moieties as well as another, longer chain ester moiety, maintaining the DS for the other moiety below a certain threshold significantly improves solubility. In some embodiments, the cellulose ester compound has an acetyl degree of substitution of less than about 1.0. In some embodiments, the acetyl degree of substitution is less than about 0.5. Embodiments also exist in which the DS for acetyl is less than about 0.8, less than about 0.6, less than about 0.4, less than about 0.3, or less than about 0.2.

It will be understood that the above values are examples useful in some embodiments and that the invention includes cellulose ester compounds having any of the values listed above (e.g. for DPᵥ, DS, DS of other esters such as acetyls) and any combination of them as long as they achieve the desired degree of solubility in the reference solvent and standard pressure and room temperature. For a given cellulose ester compound, the ideal parameters are readily determined by routine experimentation, for example by varying both DPᵥ and DS, and determining solubility for each to arrive at an optimum.

Compositions Containing the Cellulose Compounds

The compositions of the present invention contain one or more cellulose ester compounds of the present invention and one or more lipophilic solvents, with at least some of the cellulose ester compound(s) dissolved in the solvent. In some embodiments, the cellulose ester compound is dissolved at or above certain concentrations at room temperature and standard pressure. Examples include cellulose ester compounds dissolved at concentrations at least about any of the following percentages (by weight): 5%, 10%, 15%, 20%, 25%, 30%, 35%, 40%, 45%, or 50%. Solutions can be made by any effective means. In some embodiments, it is accomplished by heating the components to an appropriate temperature with stirring or other agitation. In many cases, this can be accomplished by heating up to 90° C., although higher temperatures may be necessary or useful depending on the compounds and applications involved.

The compositions are useful in oil-based cosmetic products or as part of the oil-phase in emulsions used in cosmetic or personal care products. As such, the compositions may contain any other suitable ingredients. Examples of ingredients suitable in personal care products include, for example, cleansing agents, emollients, moisturizers, pigments, including pearlescent pigments, colorants, fragrances, biocides, preservatives, antioxidants, antiperspirant agents, oral care agents, exfoliants, hormones, enzymes, medicinal compounds, vitamins, ultraviolet light absorbers, dihydroxyacetone, skin bleaching agents, antiacne agents, botanical extracts, silicone oils, organic oils, waxes, adhesion promoters, plasticizers, film formers, including hair fixatives, thickening agents, fillers and binders, alcohol and other organic solvents, and propellants. Compositions that involve emulsions can include oil-in-water emulsions, water-in-oil emulsions, as well as multiple phase emulsions, such as, for example, oil-in-water-in-oil and water-in-oil-in-water emulsions. Such emulsions may contain emulsifying agents or surfactants, for example to allow the oil phase(s) and water phase(s) to mix in such a way that one phase is continuous phase, while the other is a discontinuous phase that may be suspended in the form of micelles in the continuous phase. In such an emulsion, the oil phase may contain the cellulose ester compounds, optionally with other ingredients, while the water or aqueous phase may optionally contain other ingredients. In some skin care products, for example, aqueous phase compounds may include humectants such as glycols, sugars, and the like. Examples of suitable glycols include propylene glycol, polyethylene glycols, polypropylene glycols, and glycerin. Examples of sugars include glucose, fructose, inositol, and sucrose. Other water-soluble ingredients include gelling agents such as water-soluble or swellable gums, and water soluble polymers, including polymers of acrylic acid and esters thereof.

Although compositions useful in personal care and cosmetics are discussed above, the invention is not limited to such compositions and uses. Examples of other compositions and uses within the scope of the invention include: compositions useful as polymer additives (nucleation, clarification, impact modification, etc.); adhesives; paints and other coatings; inks; lubricants; electronics; displays; polarization films; compensation films; immobilizing compositions (including compositions for immobilizing organisms and enzymes in bioreactors); rheological modifier or thickener internally plasticized shaped articles; foods, food oils, or food-based food products, transdermal delivery of substances; membranes for various uses.

Uses of the Compositions

Any use of the compositions of the present invention is within the scope of the invention. One example is personal care products. Examples of such products include deodorants, antiperspirants, combination antiperspirant deodorants, shaving products, skin lotions, moisturizers, toners, bath products, cleansing products, hair care products, shampoos, conditioners, mousses, styling gels, hair sprays, hair dyes, hair coloring products, hair bleaches, hair waving products, hair straighteners, manicure products, nail polish, nail polish remover, nail creams, nail lotions, cuticle softeners, protective creams, sunscreen products, insect repellent, anti-aging products, color cosmetics, lipsticks, foundations, face powders, eye liners, eye shadows, blushes, makeup, mascara, personal care formulations where cellulosic components have been conventionally added, and drug delivery systems for topical application of medicinal compositions that are to be applied to the skin. As noted above, such uses are not limiting and any use is within the scope of the invention.

The present invention is illustrated in greater detail by the specific examples presented below. It is to be understood that these examples are illustrative embodiments and are not intended to be limiting of the invention, but rather are to be construed broadly within the scope and content of the appended claims. All parts and percentages in the examples are on a weight basis unless otherwise stated.

### EXAMPLES

### Comparative Example 1

Cellulose acetate nonanoate was prepared from cellulose acetate by the pyridine-acid chloride process, a process similar to that described by C. J. Malm, et al, Industrial and Engineering Chemistry, vol 43, pages 684-688, 1951.

The following reagents were added, in the following order, to a one liter, three-neck, round bottom flask, equipped with a mechanical stirrer and cold water condenser/distillation column, and placed in a silicone oil bath: 500 mL of N-methylpyrrolidinone —(C₅H₉NO), (NMP); 17 mL of pyridine —(C₅H₅N); 30 grams of oven dried, cellulose acetate cellulose acetate with an acetyl content between 31.0 and 33.0 weight %, an intrinsic viscosity in pyridine of approximately 0.88 dL/g and a weight average molecular weight (M_{w}) of approximately 47,500 Daltons, measured by size exclusion chromatography in N-methylpyrrolidinone. The cellulose acetate was prepared in a manner similar to that described in Gedon, S.; Fengl, R. “Cellulose Esters,” Kirk-Othmer Encyclopedia of Chemical Technology, 4th Ed., vol. 5, John Wiley & Sons, New York, 1993, pp. 496-529. This mixture was stirred at room temperature until the cellulose acetate was dissolved. To this mixture, 27 mL of 96% nonanoyl chloride (C₉H₁₇ClO) (Aldrich; Catalog No. 156833) was added drop-wise over approximately 30 minutes with constant stirring. The entire mixture was then warmed to 90-91° C. and stirred at this temperature for 24 hours. 35 mL of deionized water was then added to the reaction mixture to assure the decomposition of any remaining nonanoyl chloride. The resulting cellulose ester product was precipitated by stirring the reaction mixture into methanol. After several changes of methanol to wash the product free of solvents, the product was transferred to a filter bag and washed with deionized water overnight. The product was dried in a vacuum oven under a nitrogen purge for 24 hours at 50 and an additional 24 hours at 80° C. The resulting dry product was analyzed by NMR and found to contain DS acetyl of 1.7 and a DS nonanoyl of 1.5. The weight-average molecular weight (M_{w}) was determined to be 1.09×10⁵ Daltons against a polystyrene standard using gel permeation chromatography using in tetrahydrofuran.

Solubility determination: To determine solubility in various solvents, separate vials were prepared with 1% loading or 8% loading of the polymer, (by “loading” it is meant that amounts that, if dissolved, would result in 1% or 8% solutions, were each placed in a given vial). Vials were then visually inspected to determine if the polymers had dissolved, as indicated by the absence of any visible solids in the solvent. Where solids were observed, the solvents were heated and agitated to maximize the potential to dissolve. If visible solids were observed upon heating, the polymer was not considered soluble to the concentration given. The product was acetone soluble and not soluble at 1% or 8% in isohexadecane, (Creasil IH™, or isododecane, (Creasil ID™). (Creasil IH™ and Creasil ID™ are trade names of Optima Specialty Chemical LLC). The solubility was examined by placing the amounts of each polymer in separate vials containing the solvents.

### Comparative Example 2

Cellulose acetate nonanoate was prepared from cellulose acetate by the pyridine-acid chloride process.

The following reagents were added, in the following order, to a one liter, three-neck, round bottom flask, equipped with a mechanical stirrer and cold water condenser/distillation column, and placed in a silicone oil bath: 292 mL of N-methylpyrrolidone; 28 mL of pyridine; 30 grams of oven dried cellulose acetate with an acetyl content between 17.0 and 19.0 weight % and a weight average molecular weight (M_{w}), measured by size exclusion chromatography in N-methylpyrrolidinone, of approximately 20,000 Daltons, prepared in a manner similar to that described in Comparative Example 1 above. This mixture was stirred at room temperature until the cellulose acetate was dissolved. After the cellulose ester dissolved, 18 mL of solvent was distilled off to assure that any remaining water was removed from the reaction. To this mixture, 73 mL of 96% nonanoyl chloride; (C₉H₁₇ClO) (Aldrich; Catalog No. 156833) was added drop-wise over approximately 30 minutes with constant stirring. After the addition of the nonanoyl chloride, the mixture was warmed to 95° C. and stirred at this temperature for 24 hours. Deionized water (35 mL) was then added to the reaction mixture to ensure decomposition of any remaining nonanoyl chloride. The resulting product cellulose ester was precipitated by stirring the reaction mixture into 50/50 deionized water/methanol mixture. After several changes of methanol to wash the product free of solvent, the product was transferred to a filter bag and washed with deionized water overnight. The product was dissolved in acetone, precipitated and washed by the above procedure to produce a small particle precipitate. The product was dried in a vacuum oven under a nitrogen purge for 24 hours at 50 and an additional 24 hours at 80° C. The resulting dry product was analyzed by NMR and found to contain a DS acetyl of 0.7 and a DS nonanoyl of 2.4. The total measured DS was greater than 3.0, possibly because the product may contain free acid impurities. The weight-average molecular weight (M_{w}) was measured by gel permeation chromatography in tetrahydrofuran and found to be 6.5×10⁴ Daltons against a polystyrene standard. The solubility determination procedure from Comparative Example 1 was used for certain solvents. The product was acetone soluble at 8%, toluene soluble at 8%, but was insoluble in isohexadecane or isododecane at 1% or 8%, forming a solid that swelled but did not dissolve.

### Comparative Example 3

Cellulose acetate butyrate nonanoate was prepared from cellulose acetate butyrate by the pyridine-acid chloride process.

The following reagents were added, in the following order, to a one liter, three-neck, round bottom flask, equipped with a mechanical stirrer and cold water condenser/distillation column, and placed in a silicone oil bath: 438 mL of N-methylpyrrolidinone; 46 mL of pyridine; 30 grams of oven dried, cellulose acetate butyrate (CAB), having an acetyl content of approximately 4.01 weight %, a butyryl content of approximately 28.37 weight % and a hydroxyl content of approximately 1.30 weight %, a weight average molecular weight of approximately 40,600 Daltons, measured by size exclusion chromatography in N-methylpyrrolidinone. The CAB was prepared in a manner similar to that described in Gedon, S.; Fengl, R. “Cellulose Esters,” Kirk-Othmer Encyclopedia of Chemical Technology, 4th Ed., vol. 5, John Wiley & Sons, New York, 1993, pp. 496-529. The mixture was stirred at room temperature until the CAB was dissolved. After dissolution of the CAB, 30 mL of solvent was distilled off the reaction mixture. To this mixture, 81 mL of 96% nonanoyl chloride (C₉H₁₇ClO) (Aldrich; Catalog No. 156833) was added drop-wise over approximately 45 minutes with constant stirring. After the addition of the nonanoyl chloride the entire mixture was warmed to 95° C. and stirred at this temperature for 24 hours. 35 mL of deionized water was then added to the reaction mixture to assure the decomposition of any remaining nonanoyl chloride. The reaction product was a gelled mass in the reaction flask and was precipitated by feeding into 50/50 deionized water/methanol mixture, where it formed a soft precipitate that tended to reform into a mass if left to stand in the precipitation liquids. After three repetitions of the dissolution, precipitations and washing cycles, the product produced a particle precipitate. After several changes of methanol to wash the product free of solvents, the product was transferred to a filter bag and washed with deionized water overnight. The product was dried in a vacuum oven under a nitrogen purge for 24 hours at 50° C. The resulting dry product was difficult to analyze by NMR due to interference from the butyryl signal in the nonanoyl range. The weight-average molecular weight (M_{w}) was measured by gel permeation chromatography in tetrahydrofuran and found to be 1.4×10⁵ Daltons against a polystyrene standard. The solubility determination procedure from Comparative Example 1 was used for certain solvents. The product was acetone soluble even at 8% toluene soluble even at 8%, and not soluble in isohexadecane or isododecane at 1% or 8%.

### Comparative Example 4

Cellulose acetate laurate was prepared from cellulose acetate by the pyridine-acid chloride process.

The following reagents were added, in the following order, to a one liter, three-neck, round bottom flask, equipped with a mechanical stirrer and cold water condenser/distillation column, and placed in a silicone oil bath: 324 mL of pyridine and 30 grams of oven dried cellulose acetate with an acetyl content between 31.0 and 33.0 weight %, an intrinsic viscosity in pyridine of approximately 0.88 dL/g and a weight average molecular weight (M_{w}) of approximately 47,500 Daltons. This mixture was stirred at room temperature until the cellulose acetate dissolved. After dissolution of the cellulose acetate, 20 mL of solvent was distilled from the reaction mixture. To this mixture, 43 mL of 98%; lauroyl chloride (C₁₂H₂₃ClO) (Aldrich; Catalog No. 156930) was added drop-wise over approximately 30 minutes with constant stirring. After the addition of the lauroyl chloride the entire mixture was warmed to 90-91° C. and stirred at this temperature for 24 hours. 25 mL of deionized water was then added to the reaction mixture to assure the decomposition of any remaining lauroyl chloride. The resulting cellulose ester product was precipitated by feeding the reaction mixture into deionized water while stirring and after several changes of methanol to wash the product free of solvent, the product was transferred to a filter bag and washed with deionized water overnight. The product was dried in a vacuum oven under a nitrogen purge for 24 hours at 80° C. The resulting product was analyzed by NMR and found to contain DS acetyl of 1.92 and a DS laurate of 1.42. The total measured DS was greater than 3.0, possibly because the product may contain free acid impurities. The weight-average molecular weight (M_{w}) was measured by gel permeation chromatography in tetrahydrofuran and found to be 9.2×10⁴ Daltons against a polystyrene standard. The solubility determination procedure from Comparative Example 1 was used for certain solvents. The product was soluble in acetone, dichloromethane, and n-propyl acetate at 1 and 8%, partially soluble in toluene, and not soluble in isohexadecane or isododecane, acetic acid or isopropanol at 1% or 8%.

### Comparative Example 5

Cellulose acetate palmitate was prepared from cellulose acetate by the pyridine-acid chloride process.

The following reagents were added, in the following order, to a one liter, three-neck, round bottom flask, equipped with a mechanical stirrer and cold water condenser/distillation column, and placed in a silicone oil bath: 307 mL of pyridine; 21 mL of N-methylpyrrolidone; and 30 grams of oven dried cellulose acetate with an acetyl content between 31.0 and 33.0 weight %, an intrinsic viscosity in pyridine of approximately 0.88 dL/g and a weight average molecular weight (M_{w}) of approximately 47,500 Daltons, measured by size exclusion chromatography in N-methylpyrrolidinone. This mixture was stirred at room temperature until the cellulose acetate dissolved. After dissolution of the cellulose acetate, 31 mL of solvent was distilled from the reaction mixture. To this mixture, 56 mL of 98% palmitoyl chloride (C₁₆H₃₁ClO) (Aldrich; Catalog No. P78) was added drop-wise over approximately 30 minutes with constant stirring. After the addition of the palmitoyl chloride, the entire mixture was warmed to 95° C. and stirred at this temperature for 24 hours. Deionized water (25 mL) was then added to the reaction mixture to ensure the decomposition of any remaining palmitoyl chloride. The resulting product cellulose ester was precipitated by stirring the reaction mixture into deionized water and reprecipitated from an acetone solution. After several methanol washes to wash the product free of solvent, the product was transferred to a filter bag and washed with deionized water overnight. The product was Soxhlet extracted for 12 hours with methanol and was dried in a vacuum oven under a nitrogen purge for 24 hours at 80° C. The weight-average molecular weight (M_{w}) was measured by gel permeation chromatography in tetrahydrofuran and found to be 1.10×10⁵ Daltons against a polystyrene standard. The solubility determination procedure from Comparative Example 1 was used for certain solvents. The product from this example was not soluble and only slightly swelled in isohexadecane or isododecane, acetic acid or iso-propanol at 1% and 8%.

### Comparative Example 6

Cellulose stearate was prepared from a soft wood pulp with an α-cellulose content greater than 94 weight % employing the trifluoroacetic anhydride, stearic acid method as described in Morooka, T., Norimot, M., Yamada, T., Jour. Applied Polymer Science, 1984, 29, 3981).

The following reagents were added, in the following order, to a one liter, three-neck, round bottom flask, equipped with a mechanical stirrer and cold water condenser/distillation column, and placed in a silicone oil bath: 78.4 mL (117 g) of trifluoroacetic anhydride (Aldrich; Catalog No. 106232) and 194 grams of a composition containing stearic acid (J. T. Baker, available through VWR International, Catalog No. JT0340-07; Triple Pressed; N.F.; believed to contain at least 8-10% of two fatty acids with lower carbon content and one with higher carbon content). The mixture was stirred at 50° C. until the stearic acid dissolved and a mixed anhydride solution formed. To this solution, 10 grams of the wood pulp cellulose were added with stirring and the reaction mixture was held at 50° C. overnight with constant stirring. Approximately 200 mL of toluene was added to dilute the mixture. One half of this diluted mixture was precipitated into methanol.

Sulfuric acid (0.1 gram) was added to the other half of the mixture. This mixture was stirred at 50° C. for approximately 3 hours. The sulfuric acid was neutralized with magnesium acetate tetrahydrate. This reaction mixture was then precipitated into methanol.

Both parts of this experiment were washed first in deionized water and then in methanol. The product ester from both halves had a weight-average molecular weight of about 3.5×10⁶ Daltons as measured by gel permeation chromatography against a polystyrene standard. The solubility determination procedure from Comparative Example 1 was used for certain solvents. The product ester from both halves formed a hazy gel in isododecane at both 1 and 8% and a hazy dispersion in isohexadecane at both 1 and 8%.

### Example 7

The following reagents were added, in the following order, to a one liter, three-neck, round bottom flask, equipped with a mechanical stirrer and cold water condenser/distillation column, and placed in a silicone oil bath: 34.3 mL (51 g) of trifluoroacetic anhydride (Aldrich; Catalog No. 106232) and 93 grams of a composition containing stearic acid (J. T. Baker, available through VWR International, Catalog No. JT0340-07; Triple Pressed; N.F.; believed to contain at least 8-10% of two fatty acids with lower carbon content and one with higher carbon content) were stirred at 50° C. until the stearic acid dissolved and a mixed anhydride solution was formed. To this solution, 10 grams of oven dried cellulose acetate with an acetyl content between 17.0 and 19.0 weight % and a weight average molecular weight (M_{w}), measured by size exclusion chromatography in N-methylpyrrolidinone, of approximately 20,000 Daltons. Continuously stirring, the reaction mixture was held at 50° C. and allowed to react for 5 hours. The resulting product was isolated by precipitation into methanol (5× vol./vol.). The precipitated cellulose acetate stearate product was washed with methanol, then washed with deionized water, then washed again with methanol. Product was dried in a vacuum oven with a nitrogen purge at 35° C. The product had a DS stearate of 2.3 and a DS acetate of 0.8 and was soluble in both isohexadecane and isododecane at both 1 and 8%. The total measured DS was greater than 3.0, possibly because the product may contain free acid impurities. The product weight-average molecular weight (M_{w}) was measured by gel permeation chromatography in tetrahydrofuran and found to be 6.5×10⁴ Daltons against a polystyrene standard.

### Example 8

Cellulose nonanoate was prepared from wood pulp using a trifluoroacetic anhydride, nonanoic acid method.

The following reagents were added, in the following order, to a 500-mL, three-neck, round bottom flask, equipped with a mechanical stirrer and cold water condenser and placed in a silicone oil bath: 44 grams of 96% nonanoic acid (Aldrich; Catalog No. N29902; remaining 4% is believed to be 2-methyloctanoic acid) and 49 grams of trifluoroacetic anhydride (Aldrich; Catalog No. 106232). The mixture was heated at 50° C. for 1 hour to form a mixed anhydride. To this solution, 5 grams of a soft wood pulp with an α-cellulose content greater than 95 weight %, was added with stirring. The reaction mixture was held at 50° C. overnight with constant stirring. This reaction mixture was then precipitated into methanol, washed first in deionized water and then in methanol. The product was then dried at 50° C. under vacuum. The resulting cellulose nonanoate ester had a DS nonanoate of 3.0 and was soluble in isododecane at 1% and insoluble at 8%; and insoluble in isohexadecane at both 1 and 8%. The product weight-average molecular weight (M_{w}) was measured by gel permeation chromatography in tetrahydrofuran and found to be 6.3×10⁵ Daltons against a polystyrene standard.

### Comparative Example 9

Cellulose acetate nonanoate was prepared from cellulose acetate using trifluoroacetic anhydride nonanoic acid method.

The following reagents were added, in the following order, to a 500-mL, three-neck, round bottom flask, equipped with a mechanical stirrer and cold water condenser and placed in a silicone oil bath: 44 grams of 96% nonanoic acid (Aldrich; Catalog No. N29902; remaining 4% is believed to be 2-methyloctanoic acid) and 49 grams of trifluoroacetic anhydride (Aldrich; Catalog No. 106232). The mixture was heated at 50° C. for 1 hour to form a mixed anhydride. To this solution, 5 grams of cellulose acetate having an acetyl content between 17.0 and 19.0 weight % and a weight average molecular weight (M_{w}), measured by size exclusion chromatography in N-methylpyrrolidinone, of approximately 20,000 Daltons, was added with stirring and the reaction mixture was held at 50° C. overnight with constant stirring. This reaction mixture was then precipitated into methanol, washed first in deionized water and then in methanol. The precipitated and washed product was dried at 50° C. under vacuum. The resulting cellulose acetate nonanoate ester had a DS nonanoate of 2.48 and a DS acetate of 0.6 and was insoluble in isododecane and isohexadecane at both 1 and 8%. The total measured DS was greater than 3.0, possibly because the product may contain free acid impurities. The product weight-average molecular weight (M_{w}) was measured by gel permeation chromatography in tetrahydrofuran and found to be 3.9×10⁴ Daltons against a polystyrene standard.

### Examples 10-23

Compositions of the present invention, including mixed esters were prepared from cotton linters using the trifluoroacetic anhydride carboxylic acid method.

Modified cellulose compounds were prepared using the quantities of reagents shown in Table 1 below. A 500 mL, three-neck, round bottom flask was equipped with a mechanical stirrer and cold water cooled vacuum distillation apparatus and placed in a silicone oil bath. The appropriate amount and type of carboxylic acid(s) for each of the example batches was added to the flask. Where cellulose compounds having mixes of types of ester moieties (e.g. cellulose acetate nonanoate) were desired, then the appropriate amounts of two types of carboxylic acids were added. After the addition of carboxylic acid(s), the specified amount of trifluoroacetic anhydride (TFAA) (Aldrich; Catalog No. 106232) was added drop-wise with stirring. While continuously stirring, the mixture was heated to 50° C. and held at this temperature for 30 to 45 minutes to allow formation of the mixed anhydride(s). To this solution, the specified amount of cotton linter cellulose (high purity dissolving-grade cellulose isolated from commercial cotton bolls, available from Buckeye Technologies, Inc., with an average degree of polymerization greater than 3900, as measured by intrinsic viscosity, for the starting cellulose) was added and the reaction mixture was held at 50 to 52° C. for 3 to 4 hours with constant stirring until the reaction was complete. To produce a smooth solution or if gelling of the product occurred, the reaction mixture was diluted to four times its volume with tetrahydrofuran or N-methylpyrrolidone. A 50/50 w/w mixture of methanol/water was added drop-wise with rapid stirring in an amount sufficient to decompose the remaining anhydride(s) but insufficient to cause precipitation of the cellulose ester product. The solution was then cooled to ambient temperature creating a viscous smooth mixture referred to as a “dope.” To separate the modified cellulose compounds product, the dope was transferred to a separatory funnel. To one part dope a mixture of solvents (heptane:methylene chloride; 9:1 (w/w)) was added and thoroughly mixed into the dope. Then methanol was added and mixed with the dope in small portions until phase separation occurred. The mixture was allowed to stand between methanol additions. The liquid-rich phase was drained away. Methanol addition and separation of the phases were repeated until the addition of a small portion of methanol began to precipitate the product. The resulting dope from this extraction process was stirred into excess methanol to precipitate the product. The product was separated from the precipitation liquids by filtration, washed with methanol several times, and then dried overnight under vacuum with a slight nitrogen purge at 50° C. The DS, molecular weight and thermal transitions of the resulting cellulose esters were determined by NMR, GPC and DSC, respectively, and are illustrated in Table 2. In Table 2, LCE refers to the “long chain ester” (i.e. nonanoate, isostearate, stearate).

TABLE 1

| | | | | Carboxylic | Acetic |
|---|---|---|---|---|---|
| Example | | Cellulose | TFAA | Acid | Acid |
| Number | Ester Type | (grams) | (grams) | (grams) | (grams) |
| 10 | cellulose nonanoate | 15 | 134 | 137 | 0 |
| 11 | cellulose nonanoate | 15 | 134 | 137 | 0 |
| 12 | cellulose nonanoate | 15 | 134 | 137 | 0 |
| 13 | cellulose acetate | 14 | 125 | 104 | 6 |
| | nonanoate | | | | |
| 14 | cellulose acetate | 14 | 125 | 104 | 6 |
| | nonanoate | | | | |
| 15 | cellulose acetate | 14 | 125 | 104 | 6 |
| | nonanoate | | | | |
| 16 | cellulose isostearate | 15 | 134 | 200 | 0 |
| 17 | cellulose isostearate | 20 | 179 | 267 | 0 |
| 18 | cellulose isostearate | 20 | 179 | 267 | 0 |
| 19 | cellulose acetate | 20 | 179 | 231 | 8 |
| | isostearate | | | | |
| 20 | cellulose stearate | 10 | 97 | 162 | 0 |
| 21 | cellulose stearate | 10 | 89 | 133 | 0 |
| 22 | cellulose stearate | 18 | 161 | 240 | 0 |
| 23 | cellulose acetate stearate | 23 | 181 | 204 | 9 |

TABLE 2

| | | | | Molecular | Thermal | |
|---|---|---|---|---|---|---|
| | | DS | DS | Weight^{a} | Transition(s), ° C. | |
| Example | | LCE | Acetate | (by GPC) | (by DSC) | |
| Number | Ester Type | (by NMR) | (by NMR) | Mw | Tg | Tm |
| 10 | cellulose nonanoate | 2.9 | — | 6.1 × 10^{5b} | — | 112^{b} |
| 11 | cellulose nonanoate | 3.02 | — | | | |
| 12 | cellulose nonanoate | 3.04 | — | | | |
| 13 | cellulose acetate | 2.7 | 0.3 | 7.0 × 10^{5b} | 84^{b} | — |
| | nonanoate | | | | | |
| 14 | cellulose acetate | n/a | n/a | | | |
| | nonanoate | | | | | |
| 15 | cellulose acetate | n/a | n/a | | | |
| | nonanoate | | | | | |
| 16 | cellulose isostearate | 3.3 | — | 8.4 × 10⁵ | — | 121 |
| 17 | cellulose isostearate | 3.65 | — | 8.0 × 10⁶ | | 110 |
| 18 | cellulose isostearate | 3.54 | — | not tested | not tested | |
| 19 | cellulose acetate | n/a | n/a | 4.8 × 10⁶ | 89 | — |
| | isostearate | | | | | |
| 20 | cellulose stearate | n/a | n/a | 5.6 × 10⁶ | — | 32(vb)^{c} |
| 21 | cellulose stearate | n/a | n/a | 6.1 × 10⁶ | — | 35(vb)^{c} |
| 22 | cellulose stearate | n/a | n/a | 7.8 × 10⁶ | — | 51(vb)^{c} |
| 23 | cellulose acetate | 2.4 | 0.9 | 6.3 × 10⁶ | — | 26(b)^{c} |
| | stearate | | | | | |

^{a}Weight-average molecular weight in tetrahydrofuran vs. polystyrene equivalents
^{b}Samples from 3 examples were combined for analysis
^{c}vb: very broad melting peak; b: broad melting peak

Table 3 gives descriptions of mixtures resulting from mixing modified cellulose compounds (cellulose nonanoate combined equal amounts of samples from Examples 10-12) of the present invention with the specified cosmetically acceptable solvent at concentrations of 1, 2, and 4 weight %. For each mixture, the solvent and modified cellulose compounds were weighed into a small vial. The vial was capped, and rolled overnight at about 65° C. The mixtures were observed after sitting at room temperature for a period of one to three months.

In Tables 3-8, the following terms and abbreviations have the following meanings:

“cl hi-vis liq” means clear, high-viscosity liquid.

“cl med-vis liq” means clear, medium-viscosity liquid

“cl lo-vis liq” means clear low-viscosity liquid

“Hazy” refers to haziness but not the presence of solid bodies. While it is not clear whether the haze denotes a solution or a suspension, no precipitation or solid bodies were observed

“Insoluble” means that the some type of solid bodies were observed, either particles that swelled with solvent to various degrees but did not absorb all of the solvent or particles that were unaffected by the solvent.

“Gel” refers to the formation of a homogeneous gel (i.e. without solid particles or a separate liquid phase) that does not move with force of gravity. Sample can be turned upside-down and material does not flow over period of several hours. By contrast, high-viscosity liquid does flow due to gravity, but extremely slowly.

Viscosity was determined using a shear rate of 1 to 5 rad/sec with “low viscosity” being defined as less than 500 centipoise; “medium viscosity” being defined as between 500 and 2000 centipoise; and “high viscosity” being defined as greater than 2000 centipoise.

TABLE 3

| | Cellulose Nonanoate | | |
|---|---|---|---|
| Solvent | 1% | 2% | 4% |
| Isohexadecane | cl lo-vis liq | cl lo-vis liq | cl hi-vis liq |
| Isododecane | cl lo-vis liq | cl lo-vis liq | cl med-vis liq |
| mineral oil (Drakeol 9) | cl lo-vis liq | Cl med-vis liq | clear gel |
| ethylhexyl palmitate | cl lo-vis liq | cl lo-vis liq | cl hi-vis liq |
| C12-15 alkyl benzoate | not tested | not tested | cl med-vis liq |
| corn oil | cl lo-vis liq | Cl med-vis liq | cl hi-vis liq |
| isocetyl alc (Jarcol I-16) | hazy liquid | not tested | not tested |
| isostearyl alc (Jarcol I- | hazy liquid | not tested | not tested |
| 18T) | | | |
| isostearyl alc (Jarcol I- | cl lo-vis liq | cl lo-vis liq | cl med-vis liq |
| 18EX) | | | |
| octyldodecanol (Jarcol I- | hazy liquid | not tested | not tested |
| 20) | | | |
| isopalmitic acid (Jaric I- | insoluble | not tested | not tested |
| 16) | | | |
| isostearic acid (Jaric I- | cl lo-vis liq | Cl med-vis liq | cl hi-vis liq |
| 18CG) | | | |
| oleic acid (Pamolyn 100) | cl lo-vis liq | cl lo-vis liq | cl med-vis liq |
| dimethicone (DC 200) | not tested | not tested | Insoluble |
| cyclopentasiloxane (DC | insoluble | not tested | not tested |
| 245) | | | |
| 1/1 DC 245/isododecane | not tested | not tested | cl hi-vis liq |

Table 4 gives descriptions of mixtures resulting from mixing a modified cellulose compound (cellulose acetate nonanoate from combining equal amounts of samples from Ex. 13-15) of the present invention with the specified cosmetically acceptable solvent at concentrations of 1, 2, and 4 weight %. For each mixture, the solvent and modified cellulose compounds were weighed into a small vial. The vial was capped, and rolled overnight at about 65° C. The cellulose acetate nonanoate had a DS nonanoate of 2.6 and a DS acetate of 0.42. The mixtures were observed after sitting at room temperature for at least one month and up to three months.

TABLE 4

| | Cellulose Acetate Nonanoate | | |
|---|---|---|---|
| Solvent | 1% | 2% | 4% |
| Isohexadecane | insoluble | clear gel | not tested |
| Isododecane | cl lo-vis liq | clear gel | clear gel |
| mineral oil (Drakeol 9) | insoluble* | not tested | not tested |
| ethylhexyl palmitate | cl lo-vis liq | cl hi-vis liq | clear gel |
| C12-15 alkyl benzoate | Not tested | not tested | cl med- |
| | | | vis liq |
| corn oil | cl med- | cl med- | cl hi- |
| | vis liq | vis liq | vis liq |
| isocetyl alc (Jarcol I-16) | cl lo-vis liq | cl hi-vis liq | hazy gel |
| isostearyl alc (Jarcol I-18T) | insoluble | not tested | not tested |
| isostearyl alc (Jarcol I-18EX) | cl lo-vis liq | clear gel | not tested |
| octyldodecanol (Jarcol I-20) | hazy liquid | not tested | not tested |
| isopalmitic acid (Jaric I-16) | cl lo-vis liq | cl hi-vis liq | clear gel |
| isostearic acid (Jaric I-18CG) | cl lo- | cl med- | cl hi vis liq |
| | vis liq | vis liq | |
| oleic acid (Pamolyn 100) | cl lo-vis liq | cl lo-vis liq | cl hi-vis liq |
| dimethicone (DC 200) | Not tested | not tested | Insoluble |
| cyclopentasiloxane (DC 245) | insoluble | not tested | not tested |
| 1/1 DC 245/isododecane | Not tested | not tested | Insoluble |

*gel layer on bottom

Table 5 gives descriptions of mixtures resulting from mixing a modified cellulose compound cellulose isostearate made by combining equal amounts of samples from Ex. 16 & 17 of the present invention with the specified cosmetically acceptable solvent at concentrations of 1, 2, and 4 weight %. For each mixture, the solvent and modified cellulose compounds were weighed into a small vial. The vial was capped, and rolled overnight at about 65° C. The mixtures were observed after sitting at room temperature for 1 to 3 months.

TABLE 5

| | Cellulose Isostearate | | |
|---|---|---|---|
| Solvent | 1% | 2% | 4% |
| Isohexadecane | cl lo-vis liq | cl lo-vis liq | clear gel |
| Isododecane | cl lo-vis liq | cl lo-vis liq | cl hi vis liq |
| mineral oil (Drakeol 9) | insoluble | not tested | not tested |
| ethylhexyl palmitate | cl lo-vis liq | cl lo-vis liq | cl med- |
| | | | vis liq |
| C12-15 alkyl benzoate | Not tested | not tested | cl med- |
| | | | vis liq |
| corn oil | cl lo vis liq | cl lo vis liq | insoluble* |
| isocetyl alc (Jarcol I-16) | cl lo-vis liq | cl med- | cl hi-vis liq |
| | | vis liq | |
| isostearyl alc (Jarcol I-18T) | cl lo-vis liq | cl med- | cl hi-vis liq |
| | | vis liq | |
| isostearyl alc (Jarcol I-18EX) | cl lo-vis liq | cl med- | insoluble* |
| | | vis liq | |
| octyldodecanol (Jarcol I-20) | cl lo-vis liq | cl lo/med- | cl hi-vis liq |
| | | vis liq | |
| isopalmitic acid (Jaric I-16) | cl lo-vis liq | cl hi-vis liq | clear gel |
| isostearic acid (Jaric I-18CG) | cl lo-vis liq | cl med- | insoluble* |
| | | vis liq | |
| oleic acid (Pamolyn 100) | cl lo-vis liq | cl lo-vis liq | insoluble |
| dimethicone (DC 200) | Not tested | not tested | insoluble |
| cyclopentasiloxane (DC 245) | insoluble | not tested | not tested |
| 1/1 DC 245/isododecane | Not tested | not tested | clear gel |

*Gel layer on bottom

Table 6 gives descriptions of mixtures resulting from mixing a modified cellulose compound (cellulose acetate isostearate sample from Example 19) of the present invention with the specified cosmetically acceptable solvent at concentrations of 1, 2, and 4 weight %. For each mixture, the solvent and modified cellulose compounds were weighed into a small vial. The vial was capped, and rolled overnight at about 65° C. The mixtures were observed after sitting at room temperature for 1 to 3 months.

TABLE 6

| | Cellulose Acetate Isostearate | | |
|---|---|---|---|
| Solvent | 1% | 2% | 4% |
| Isohexadecane | insoluble | not tested | not tested |
| Isododecane | insoluble | not tested | not tested |
| mineral oil (Drakeol 9) | insoluble | not tested | not tested |
| ethylhexyl palmitate | insoluble | clear gel | not tested |
| C12-15 alkyl benzoate | Not tested | not tested | clear gel |
| corn oil | insoluble | insoluble | not tested |
| isocetyl alc (Jarcol I-16) | insoluble | not tested | not tested |
| isostearyl alc (Jarcol I-18T) | insoluble | not tested | not tested |
| isostearyl alc (Jarcol I-18EX) | insoluble | not tested | not tested |
| octyldodecanol (Jarcol I-20) | insoluble | not tested | not tested |
| isopalmitic acid (Jaric I-16) | insoluble | not tested | not tested |
| isostearic acid (Jaric I-18CG) | insoluble | insoluble | insoluble |
| oleic acid (Pamolyn 100) | insoluble | clear gel | not tested |
| dimethicone (DC 200) | Not tested | not tested | not tested |
| cyclopentasiloxane (DC 245) | insoluble | not tested | not tested |
| 1/1 DC 245/isododecane | Not tested | not tested | not tested |

Table 7 gives descriptions of mixtures resulting from mixing a modified cellulose compound (cellulose stearate made by combining equal amounts of samples from Ex. 20-22) of the present invention with the specified cosmetically acceptable solvent at concentrations of 1 and 4 weight %. For each mixture, the solvent and modified cellulose compounds were weighed into a small vial. The vial was capped, and rolled overnight at about 65° C. The mixtures were observed after sitting at room temperature for 1 to 3 months.

TABLE 7

Cellulose Stearate

| Solvent | 1% | 4% |
|---|---|---|
| Isohexadecane | insoluble | not tested |
| Isododecane | insoluble | not tested |
| Mineral oil (Drakeol 9) | insoluble | not tested |
| ethylhexyl palmitate | hazy liq | not tested |
| C12-15 alkyl benzoate | not tested | hazy gel |
| corn oil | insoluble | not tested |
| Isocetyl alc (Jarcol I-16) | insoluble | not tested |
| isostearyl alc (Jarcol I-18T) | insoluble | not tested |
| isostearyl alc (Jarcol I-18EX) | insoluble | not tested |
| octyldodecanol (Jarcol I-20) | insoluble | not tested |
| isopalmitic acid (Jaric I-16) | insoluble | not tested |
| isostearic acid (Jaric I-18CG) | insoluble | not tested |
| oleic acid (Pamolyn 100) | insoluble | not tested |
| dimethicone (DC 200) | not tested | not tested |
| cyclopentasiloxane (DC 245) | insoluble | not tested |
| 1/1 DC 245/isododecane | not tested | not tested |

Table 8 gives descriptions of mixtures resulting from mixing a modified cellulose compound (cellulose acetate stearate sample from Example 23) of the present invention with the specified cosmetically acceptable solvent at concentrations of 1, 2 and 4 weight %. For each mixture, the solvent and modified cellulose compounds were weighed into a small vial. The vial was capped, and rolled overnight at about 65° C. The mixtures were observed after sitting at room temperature for 1 to 3 months.

TABLE 8

| | Cellulose Acetate Stearate | | |
|---|---|---|---|
| Solvent | 1% | 2% | 4% |
| Isohexadecane | insoluble | not tested | not tested |
| Isododecane | insoluble | not tested | not tested |
| mineral oil (Drakeol 9) | insoluble | not tested | not tested |
| ethylhexyl palmitate | insoluble | not tested | not tested |
| C12-15 alkyl benzoate | not tested | not tested | clear gel |
| corn oil | insoluble | not tested | not tested |
| isocetyl alc (Jarcol I-16) | insoluble | not tested | not tested |
| isostearyl alc (Jarcol I-18T) | insoluble | not tested | not tested |
| isostearyl alc (Jarcol I-18EX) | insoluble | not tested | not tested |
| octyldodecanol (Jarcol I-20) | insoluble | not tested | not tested |
| isopalmitic acid (Jaric I-16) | insoluble | not tested | not tested |
| isostearic acid (Jaric I-18CG) | insoluble | clear gel | clear gel |
| oleic acid (Pamolyn 100) | insoluble | clear gel | not tested |
| dimethicone (DC 200) | not tested | not tested | not tested |
| cyclopentasiloxane (DC 245) | insoluble | not tested | not tested |
| 1/1 DC 245/isododecane | not tested- | not tested | not tested |

### Comparative Example 24

Preparation of Isostearoyl Chloride:

80.1 grams of Isostearic Acid from A& E Connock, Perfumery and Cosmetics, LTD (a complex mixture of methyl-branched and straight-chain C18 fatty acids) was added to a round bottom flask, equipped with a condenser type distilling head, mechanical stirrer, and a thermostatically-controlled oil bath. The initial temperature of the bath was about 25° C. Over a time period of 50 minutes, thionyl chloride (39 grams, 0.33 moles) (REAGENTPLUS, Aldrich Catalog No. 230464) was added drop-wise to the isostearic acid, with constant stirring. At about halfway through the addition the oil bath temperature was raised to 35° C. and the reaction was stirred for an additional 2 hours. Vacuum was applied (90 mm Hg) to the distillation column and the oil bath temperature was increased to 50° C. Unreacted thionyl chloride (4.5 grams) was distilled away from the product yielding 85 grams of isostearoyl chloride, which was used without further purification.

A specimen of cellulose acetate butyrate having isostearyl ester groups was attempted to be prepared following the procedure described in Example 1 of the PCT International Patent Application No. PCT/FR2004/001202 (Publication No. WO 2005/013926). The batch size was 25% of that disclosed in Example 1 of the WO2005/013926 publication. The reagents were added, in the following order to a room temperature, 1000 mL, three-neck, round bottom flask equipped with a mechanical stirrer, cold water cooled condenser, vented to a drying tube filled with anhydrous calcium sulfate, a dry nitrogen inlet tube, and placed in a silicone oil bath:

225 grams of toluene (Burdick and Jackson—B&J High Purity Solvent grade); and

225 grams of methyl ethyl ketone (Mallinckrodt—analytical reagent grade).

Slowly with rapid stirring, 25 grams of cellulose acetate butyrate (Eastman Chemical Company CAB-553-0.4, 46.43 weight % butyryl) was added and dissolved by heating to 50° C. with continual stirring for 1 hour. The mixture was cooled to room temperature and 5.0 grams of triethylamine (REAGENTPLUS, Aldrich Catalog No. 90340) is added to the mixture. With continual stirring and under a dry nitrogen atmosphere the flask was cooled in an ice bath to +5° C. When the mixture reached a temperature of +5° C., 14.22 grams of the isostearoyl chloride prepared above (dissolved in 25 grams of toluene and 25 grams of methyl ethyl ketone) was added drop-wise from an addition funnel over 1 hour and 30 minutes. The temperature was measured several times during addition with a hand held electronic thermometer. Maximum temperature reached in the reaction mixture was 7.1° C. The reaction mixture was removed from the ice bath and returned to room temperature (22° C.) and held with constant slow stirring for 18 hours. Crystals believed to be triethylamine hydrochloride clouded the reaction mixture but were not seen on the sides of the flask.

The resulting mixture was filtered with a medium fritted glass funnel and then through filter paper. Portions of the reaction mixture were individually stirred into methanol, ethanol and isopropanol. No filterable precipitate formed; only a hazy yellow solution or milky solution formed with no filterable precipitate. The balance of the reaction mixture was stirred into methanol/water, 50/50, w/w, forming a milky solution and a greasy, yellow liquid phase was formed that contained a small amount of precipitate. The recovered precipitate was found to be isostearic acid resulting from the decomposition of unreacted isostearoyl chloride with water during the precipitation process, so no sample of a solidified polymer was isolated. Therefore, the expected solid precipitate of cellulose acetate butyrate isostearate was not successfully produced using this methodology.

### Example 24A

Specimens of cellulose acetate butyrate isostearate were prepared by the procedure described in Example 1 of the International Patent Application No. PCT/FR2004/001202 (Publication No. WO 2005/013926), except the product was precipitated in heptane instead of alcohol. Two batches were prepared; each batch size was 25% of that disclosed in Example 1 of the WO2005/013926 publication. The reagents were added, in the following order to each of two 1000 mL, three-neck, round bottom flasks, equipped with stirrers, cold water cooled condensers vented to drying tubes filled with anhydrous calcium sulfate, and dry nitrogen inlets, and placed in silicone oil baths:

225 grams of toluene per flask—Burdick and Jackson—B&J High Purity Solvent grade

225 grams of methyl ethyl ketone per flask—Mallinckrodt—analytical reagent grade

With rapid stirring, 25 grams of cellulose acetate butyrate (Eastman Chemical Company CAB-553-0.4-46.43 weight % butyryl) was slowly added to each flask and dissolved by heating to 50° C. with continual stirring for 1 hour. The mixtures were cooled to room temperature and 7.5 grams of triethylamine (REAGENTPLUS, Aldrich Catalog No. 90340) was added to each flask (excess triethylamine to prevent HCL degradation of the product ester). With continual stirring and under a dry nitrogen atmosphere the flasks were cooled in an ice bath to +5° C.

When the mixtures reached +5° C., 20.00 grams of the isostearoyl chloride prepared above (dissolved in 25 grams of toluene and 25 grams of methyl ethyl ketone) was added drop-wise from addition funnels to each flask over approximately 1 hour and 30 minutes. The temperature of the flasks was measured several times during addition using a hand held electronic thermometer. Maximum temperature reached in the reaction mixtures was 7.6° C. The reaction mixtures were removed from the ice bath and returned to room temperature (22° C.) and held with constant slow stirring for 18 hours. Crystals believed to be triethylamine hydrochloride clouded the reaction mixtures but were not seen on the sides of the flasks.

The resulting mixtures from both flasks were combined, filtered first using a medium fritted glass funnel, then filtered through filter paper, and precipitated in heptane. The mixture made a white flaky precipitate that was large and solid enough to filter from the precipitation liquids. The precipitate was washed twice in heptane and dried to a constant weight under nitrogen and vacuum at 55° C. The recovered precipitate weighed 46.96 grams and had a DS for isostearate of 0.17 by NMR spectral analysis. The product had a weight-average molecular weight of 3.2×10⁴ Daltons as measured by gel permeation chromatography against a polystyrene standard. The product was insoluble in isododecane and isohexadecane.

### Examples 25-27

Various solvents were investigated to determine which would solubilize cellulose nonanoate (CN), cellulose acetate nonanoate (CAN), and cellulose isostearate (CIS) at the highest concentration, with the intention of making drawdowns of the solutions and drying to produce films. The CN used was a mixture of equal amounts of product from Examples 10-12. The CAN used was a mixture of equal amounts of product from Examples 13-15. The CIS used was a mixture of equal amount of product from Examples 16 and 17.

Solvents tested were methyl acetate, butyl acetate, n-methylpyrrolidone, mineral spirits, cyclohexanone, isophorone, methanol, and Aromatic 100 hydrocarbon fluid (ExxonMobil). Aromatic 100 hydrocarbon fluid was found to be most suitable, solubilizing all three modified cellulose compounds at 15 weight %. The other solvents would not solubilize the modified cellulose compounds at concentrations greater than 10 weight %. Solutions of the specified modified cellulose compounds were made at 15 weight % in Aromatic 100 fluid. Drawdowns of the solutions were made on to metal plates and Leneta chart paper. The films were dried at room temperature. The resulting films were clear having an average thickness of about 2 mils. The films were tested for Pendulum hardness (ASTM D4366-87), Tukon hardness (ASTM D1474), gloss, and flexibility by a flex bar test and tactile evaluation. Results are given in Table 9 below. Each test result is the average of 3 measurements.

TABLE 9

| | CN | CAN | CIS |
|---|---|---|---|
| Pendulum Hardness, | 42 | 62 | 8 |
| sec | | | |
| Tukon Hardness, | 1.0 | 1.5 | <0.5 |
| knoops | | | |
| Flex Bar Test | No break thru | No break thru | No break thru |
| | ⅛ inch | ⅛ inch | ⅛ inch |
| Flexibility Ranking* | 2 | 3 | 1 |
| Gloss Units | 80 | 83 | 83 |
| Refractive Index | 1.473 | 1.472 | 1.476 |

*1 is the softest and most flexible, 3 is the hardest and least flexible. All three films had a good gloss, and can contribute gloss to a personal care product if desired.

### Examples 28-31

In each of the following examples, a personal care product, (mascara) was prepared using CN, CAN, or CIS dissolved in a suitably cosmetically acceptable solvent. The CN, CAN and CIS were the same mixed products described for EXAMPLES 25-27 above. The specified amounts of wax, stearic acid, ethylhexyl palmitate, and the modified cellulose compounds were weighed into a beaker and heated to 80° C. The ingredients were mixed when melted to obtain a homogeneous mixture. The gum arabic was added to the water and allowed to hydrolyze overnight at room temperature. The water/gum mixture was heated to 50° C. with stirring while slowly adding the hydroxyethylcellulose, followed by triethanolamine. The aqueous phase was heated to 80° C.; then the wax phase was slowly added to the aqueous phase while mixing. The mixture was cooled to 40° C. and the preservative was added. Mixing was continued until blended. The results are presented in Table 10 below.

TABLE 10

| | Example | | Comparative | | Comparative | | Example | |
|---|---|---|---|---|---|---|---|---|
| Ingredient | 28 | | Ex. 29 | | Ex. 30 | | 31 | |
| Candelilla wax | 4.9 | g | 4.9 | g | 4.9 | g | 3.1 | g |
| Paraffin wax | 4.3 | | 4.3 | | 4.3 | | 2.7 | |
| Beeswax | 9.2 | | 9.2 | | 9.2 | | 5.8 | |
| Stearic acid | 6.3 | | 6.3 | | 6.3 | | 4.0 | |
| Ethylhexyl | 10.8 | | 10.8 | | 10.8 | | 6.8 | |
| palmitate | | | | | | | | |

| | | | | |
|---|---|---|---|---|
| CN | 5.4 | — | — | — |

| | | | | |
|---|---|---|---|---|
| CAN | — | 5.4 | — | — |

| CIS | — | — | 5.4 | 3.4 |
|---|---|---|---|---|
| Water | 51.5 | — | 51.5 | 64.7 |
| Gum Arabic | 3.7 | — | 3.7 | 4.6 |
| Hydro- | 1.0 | — | 1.0 | 1.3 |
| xyethylcellulose | | | | |
| Triethanolamine | 2.6 | — | 2.6 | 3.3 |
| Preservative | 0.3 | — | 0.3 | 0.3 |
| (Germaben II) | | | | |

The example with CN had a creamy consistency. When applied to eyelashes, it separates and defines the lashes. The formulation given as the comparative example with CAN was not completed because the CAN did not dissolve in the melted wax phase ingredients. The first example with CIS formed a solid, not suitable for use as a mascara. The second formulation with CIS with a reduced wax phase concentration has a creamy consistency. When applied to eyelashes, it separates and defines the lashes. The formulation of this example was also applied and spread on the skin. It spread easily, felt smooth as it was spread, and left a water-resistant film on the skin. As is known in the art, pigments may be added to the above formulations.

### Example 32

In the following example, a personal care product, (lipstick) was prepared using CIS (a mixture of equal amount of product from Examples 16 and 17) in a suitably cosmetically acceptable solvent. The ingredients were weighed into a jar and placed in an oven at 95° C. When all ingredients had melted, they were mixed until homogeneous. As the mixture cooled it was poured into a lipstick mold. The resulting lipstick was evaluated by applying to the skin. The specified amounts and the ingredients are presented in Table 11 below.

TABLE 11

| Ingredient: | Weight % |
|---|---|
| Polyethylene wax | 30.8 |
| CIS | 4.7 |
| Isododecane | 46.1 |
| Hydrogenated polyisobutene | 7.7 |
| Isostearyl alcohol | 2.8 |
| Phenyl trimethicone | 3.6 |
| C30-39 olefin/isopropylmaleate/MA | 0.9 |
| Copolymer | |
| Pigment Wax Dispersion | 3.2 |

The stick had poor glide, but deposited the color well. A few minutes after application, it felt dry and was not greasy. Color adhered well; would not rub off.

### Examples 33-35

In the following examples, hair styling products were prepared. Hair tresses were purchased from International Hair Importers & Products, Inc., Floram Park, N.Y., made to the following specifications: virgin, medium brown hair; 2.2 grains hair per tress; 20 cm long. Before testing all tresses are hung on a peg board and cut to precisely 6½ inches (26-unit mark on the peg board). Tresses were then prepared by combing, wetting, and removing excess water. An equal amount (0.2 g) of each solution or gel of 4% CN, CAN and CIS (from the same mixtures described above for Examples 25-27) in isododecane were applied to hair tresses weighing about 2.8 g by working the solution or gel through the hair with the fingers. The tresses were combed after applying the solution or gel and allowed to air dry overnight. After drying, a curling iron was used to curl the hair tresses and hung on peg board after treatment at 90% RH for 2 hours. Length of curls was then compared for hair tresses. Compared to the untreated hair tress, the modified cellulose compounds treated tresses were easier to comb, had more shine, and better curl retention under 90% relative humidity at 22° C. CN and CIS provided more gloss and better hold compared to CAN. CIS provided a flexible hold, while CN provided a stiffer hold. After application the tresses were washed with shampoo. The modified cellulose compounds were difficult to wash from the hair. Remaining modified cellulose compounds could be seen on the hair as white specks after the hair had dried.

As indicated by the poor wash-out, it is apparent that the modified cellulose compounds have good substantivity to the hair and therefore have utility as temporary hair dyes. A hair dye is incorporated into the solution of modified cellulose compounds in isododecane and applied to the hair.

To produce a low-VOC product that meets low-VOC regulations, isohexadecane was used in place of isododecane. The tresses required more time to dry and retained their oily feel after a few hours at room temperature. However, treatment with heat, for example with a hair drier or curling iron, quickly removed the isohexadecane solvent leaving behind a glossy finish, good manageability, and curl retention.

### Example 36

A sun protection product was prepared having the composition specified in Table 12 below. CN was a blend of equal amounts of the products from Examples 10-12.

TABLE 12

| Ingredient | Weight % |
|---|---|
| C12-15 alkyl benzoate (Finsolv TN) | 5.0 |
| Ethylhexyl methoxycinnamate | 7.0 |
| Butyloctyl salicylate | 2.0 |
| Octocrylene | 2.0 |
| Benzophenone-3 | 4.0 |
| Butyl methoxydibenzoylmethane | 2.0 |
| Cetearyl alcohol (and) dicetyl phosphate | 2.0 |
| (and) ceteth-10 phosphate | |
| Myristyl myristate | 3.0 |
| Glyceryl stearate | 1.0 |
| Cellulose Nonanoate | 1.0 |
| Polyester-5 | 3.0 |
| Water | 68.0 |
| Triethanolamine | 0.1 |

The oil phase ingredients and water phase ingredients were mixed separately at 80° C., then combined and mixed with a high shear mixer for 10 minutes. The resulting low-viscosity emulsion had a smooth feel when applied to the skin and left behind a water-resistant film.

### Examples 37 & 38

An antiperspirant product was prepared having the composition (weight %) specified in Table 13 below. The ingredients, except for the aluminum/zirconium tetrachlorohydrex-Gly (AAZG-7167, Summit Research Lab), were weighed into a beaker and heated with stirring to 85° C. When the mixture appeared homogeneous, the tetrachlorohydrex-Gly was added and dispersed using a high-speed disperser. The formulation was mixed for 10 minutes at 82° C., then allowed to cool. When the temperature reached 60° C., the mixture was poured into an antiperspirant stick container. After sitting overnight at room temperature the antiperspirant sticks were evaluated. Both set up to form white opaque sticks. When applied to the skin, both have a smooth feel. CIS was a blend of equal amounts of the products from Examples 16 and 17.

TABLE 13

| | | Comparative |
|---|---|---|
| Ingredient | Example 37 | Example 38 |
| Cetearyl alcohol | 20.0 | 20.0 |
| Cyclopentasiloxane | 26.5 | 30.0 |
| Isododecane | 26.0 | 26.0 |
| CIS | 3.5 | — |
| Aluminum/zirconium | 24.0 | 24.0 |
| tetrachlorohydrex-Gly | | |

Example 37 was observed to deposit more material on the skin than Comparative Example 38. After drying the CIS formulation provided a more comfortable feel on the skin, with no sensation of skin tightening. With very hard rubbing, Example 37 rolled up off of the skin indicating a film had been left behind, whereas the sample without CIS did not have this effect.

### General Discussion for Examples 39-42

Compositions of the present invention, including mixed esters were prepared from cotton linters using the trifluoroacetic anhydride carboxylic acid method.

Modified cellulose compounds were prepared via detailed preps provided below. The appropriate amount and type of carboxylic acid(s) for each of the example batches was added to the flask. Where cellulose compounds having mixes of types of ester moieties (e.g. cellulose acetate nonanoate) were desired, then the appropriate amounts of two types of carboxylic acids were added. After formation of the solution, the specified amount of cotton linter cellulose (high purity dissolving-grade cellulose isolated from commercial cotton bolls available from Buckeye Technologies, Inc.; with an average degree of polymerization, DP, greater than 1300, as measured by intrinsic viscosity) was added to the reaction mixture at the specified temperature, gradually ramped to 50 to 55° C., and held at this point with constant stirring until reaction was complete. Upon completion of reaction, the resulting smooth mixture, also referred to as a “dope”, was isolated by precipitation into 100% methanol under high shear conditions. Analytical and solubility results for Examples 39-42 are provided in Tables 14-19.

### Example 39

### Synthesis and Purification of Cellulose Stearate

Preparation of the mixed stearic acid/tri-fluoroacetic acid anhydride. To a flame dried, argon-purged 1000 mL 4-Neck round-bottom flask; equipped with a stirrer bearing/mechanical stir shaft designed for vacuum applications, a PTFE stopper (24/40 standard taper joint) with extracting nut, and two Claisen adapters that were fitted with the following: a) reflux condenser; b) 125 mL addition funnel with metering stopcock c) internal thermometer/thermocouple; was added stearic acid (166.75 g, 0.5862 moles) (J. T. Baker; Catalog No. JT0340-07; Lot # B0336; Triple Pressed; N.F; 90-100%; stearic acid; by Liquid Chromatography, material appears to contain at least 8-10% of two fatty acids with lower carbon content and one with higher carbon content). While still at room temperature and in order to completely dissolve the fatty acid, 150 mL of n-heptane (Burdick & Jackson; Catalog No. 207-4) and 50 mL of dichloromethane (Burdick & Jackson; Catalog No. 300-4) were added with stirring and mixed until a homogenous solution was achieved. In order to minimize color of the mixed anhydride, the solution was cooled to 10-15° C. and held for 30 minutes. Trifluoroacetic anhydride (TFAA) (111.92 g, 0.5329 moles; 74.07 mL) (Aldrich; Catalog No. 106232) was quickly added to a 125 mL addition funnel equipped with a fine-control metering stopcock. The glass stopper was returned to the addition funnel; and it was ensured that the positive pressure flowing through the reactor was at the lowest level possible (slowest possible bubble rate in an oil bubbler filled with silicone oil). TFAA was added to the cooled solution at a moderate rate—the rate of addition was such as to maintain the internal temperature of the reaction mixture below 15° C. After complete addition of trifluoroacetic anhydride, the cooling bath was removed and replaced with a bath containing tap water at room temperature. After 25-30 minutes (or when the internal temperature of the flask returned to 25° C.), the mixture was stirred for an additional 30 minutes to ensure that the mixed anhydride was formed. At this point, the reaction mixture was homogeneous and had developed a very light, golden-yellow hue.

Reaction of cellulose with the mixed an hydride of stearic acid and tri-fluoroacetic acid—Torn chips of linter grade cellulose (approximately 15 mm×15 mm in size; available from Buckeye Technologies, Inc.) were pre-dried at 55° C. in a vacuum oven, <25 inch Hg, for no less than 24 hours. After this period, the cellulose was quickly transferred to a 0% humidity dessicator in order to cool and was stored until use. The PTFE stopper was removed and 15.16 g, (0.0935 moles) of dried hand-torn chips were added as quickly as possible to the reaction mixture via a wide-mouth powder funnel—it was ensured that there was a minimal positive argon pressure flowing through the system when performing this operation in order to minimize vapor release from the reaction vessel. After complete addition of cellulose, the stopper was returned; and the reaction mixture was heated incrementally, using a temperature-controlled oil bath, over the next 2 hours to achieve a final internal solution temperature of 50° C. This mixture was held under these conditions with constant stirring for 5 hours. At this time the mixture had fully reacted to yield a translucent dope that was a light, golden-brown hue. Heating of the mixture was ceased after this period.

Neutralization—After cooling the constantly stirred dope to an internal temperature of 30° C., the residual trifluoroacetic anhydride was neutralized via slow, drop-wise addition of a 50:50 (w/w) mixture (based on moles in relation to excess TFAA) of methanol/Millipore water (3.80 g and/or 4.80 mL MeOH & 2.14 g H₂O) over a period of 90 minutes. In this case, the internal temperature rose to a maximum of 41.5° C. (11.5° C. increase)—Important: the rate of addition should be such as to maintain the internal temperature at or below 50° C. to minimize possible degradation of cellulose. After the internal temperature returned to 30-35° C., the neutralization was determined to be essentially complete. The reaction vessel was then prepared for distillation.

Distillation—Before distillation, both Claisen adapters, the thermocouple/thermometer adapter, the addition funnel and condenser were removed and replaced with glass stoppers lubricated with DOW Corning High Vacuum Grease. A 90° hose connection adapter was added to open neck of the 4-Neck, 1000 mL round-bottom flask and was connected in series, via high vacuum hose and Schlenk manifold, to two 500 mL vacuum traps. Both traps were cryogenically cooled with liquid nitrogen. While maintaining the internal temperature of the mixture at 30° C., vacuum was slowly/incrementally introduced until reaching an ultimate vacuum of 60 mmHg. After equilibrating at this pressure, distillation of residual trifluoroacetic acid, trifluoroacetic acid methyl ester, water and methanol was initiated by heating the crude mixture—the heat was slowly ramped as to induce/maintain a steady distillation. Heating was continued until the internal temperature reached a maximum of 50° C. With constant agitation, full vacuum was maintained at 50° C. for a period of 60 minutes. At this time, the distillation of the vast majority of lower boiling contaminants and a great deal of both dichloromethane and n-heptane were removed. Heating was ceased, vacuum terminated, and the system was backfilled with argon. The resultant mixture was a very viscous dope that had manifested a darker, amber color.

Precipitation/Isolation—In order to prepare for precipitation, 30 mL of trichloroethylene (Aldrich; Catalog No. 372145; anhydrous) was added and stirred into the resultant dope for a period of 30 minutes. The dope was transferred from the parent reactor to a separatory funnel, which was then used to slowly meter the crude dope into a vigorously stirred/highly agitated solution of 100% methanol (Burdick & Jackson; Catalog No. 230-4; 99.9+%)—the ratio for the total amount of methanol required to effectively precipitate the dope was on the order of 20-25:1 [(v/v); MeOH/dope]. After precipitation was complete, the off-white, flocculent precipitate was left to stir slowly in the precipitation vessel for an additional 60-120 minutes in order to harden the material. The precipitate was captured via vacuum filtration through a course, glass-fritted funnel. The solid was immediately transferred to an appropriately-sized flask and slurried with 2000 mL of methanol. The resultant slurry was stirred for an additional hour and left to settle and further harden for a period of no less than 12 hours. The isolated polymer was captured via vacuum filtration through a course, glass-fritted funnel; left in the funnel with vacuum still applied to air-dry for at least 60 minutes; and transferred to a drying dish in preparation for further drying. The air-dried material was further dried in a vacuum oven, <25 inch Hg, at 50° C. for no less than 24 hours. The material was removed from the vacuum oven and transferred to a dessicator in order for the polymer to cool prior to obtaining samples for analytical analysis and/or solubility testing. The reaction to produce cellulose stearate (and/or cellulose tri-O-stearate) yielded 81.54 g (90.7% yield, isolated) of waxy solid that was an off-white-to-light cream in color.

NMR procedures and related calculations were conducted in a manner so as to minimize the possibility that NMR results do not include moles residual reactants as part of the moles of ester. The solution is then allowed to cool to room temperature and at least 0.1 ml of trichloroacetyl isocyanate is added to the solution, followed by stirring for at least 5 minutes. Samples are then subjected to proton NMR spectrum (at 80° C.) within a few hours of preparation using the normal proton NMR parameters, but with 64 scans. The resulting NMR data provides a peak for free acid present as well as a peak for total acid (acids and esters combined). Both peaks are integrated and the peak for free acid is subtracted from the total acid peak to provide a value for total esterified acid. This value is used to calculate DS. With this method, an internal check is available by comparing the moles for reacted hydroxyls of the cellulose (using the hydroxyl peak) since the derivatizing agent reacts with both the unesterified hydroxyls of the cellulose and the unesterified acid. The moles of hydroxyl is calculated and divided by the moles of backbone to yield a value for number of remaining hydroxyl groups per anhydroglucose. The value 3.00 minus this value hydroxyl should yield the same DS value for esterified acid as calculated using the other method. The material was found by ¹H NMR to possess a stearate DS of 2.81. The polymer was soluble in both isohexadecane and isododecane. The protocol for solubility determination set forth above in Comparative Example 1 was conducted in isohexadecane and isododecane at the concentrations in the tables set forth below. Results were recorded after a period of 2 up to one month. Tables provide detailed analytical and solubility results.

### Example 40

### Synthesis and Purification of Cellulose Neo-Decanoate

Preparation of the mixed neo-decanoic acid/tri-fluoroacetic acid anhydride—To a flame dried, argon-purged 1000 mL 4-Neck round-bottom flask; equipped with a stirrer bearing/mechanical stir shaft designed for vacuum applications, a PTFE stopper (24/40 standard taper joint) with extracting nut, and two Claisen adapters that were fitted with the following: a) reflux condenser b) 125 mL addition funnel with metering stopcock c) internal thermometer/thermocouple; was added 100.97 g of Neo-Decanoic Acid Prime, CAS# 26896-20-8, a clear, colorless liquid at room temperature/pressure available from ExxonMobil Chemical Company, (¹H NMR, ¹³C NMR, and Liquid Chromatography (LC) provide evidence for a multi-component mixture consisting of greater than 6 compounds). In order to minimize color of the mixed anhydride, the solution was cooled to 0° C. and held for 60 minutes. Trifluoroacetic anhydride (TFAA) (111.92 g, 0.5329 moles; 74.07 mL) (Aldrich; Catalog No. 106232) was quickly added to a 125 mL addition funnel equipped with a fine-control metering stopcock. The glass stopper was returned to the addition funnel, and it was ensured that the positive pressure flowing through the reactor was at the lowest level possible (slowest possible bubble rate in an oil bubbler filled with silicone oil). TFAA was added to the cooled solution at a moderate rate over the course of between 120-150 minutes (2.0-2.5 hours)—the rate of addition was such as to maintain the internal temperature of the reaction mixture below 5° C. Interestingly, during TFAA addition, the solution began to take on a pinkish purple to reddish purple hue. After complete addition of trifluoroacetic anhydride, the cooling bath was removed and replaced with a bath containing tap water at room temperature. After 25-30 minutes (or when the internal temperature of the flask returned to 25° C.), the mixture was stirred for an additional 60 minutes at room temperature to ensure that the mixed anhydride was formed. At this point, the reaction mixture was homogeneous and had developed a very brilliant reddish purple hue.

Reaction of cellulose with the mixed anhydride of neo-decanoic acid and tri-fluoroacetic acid—Torn chips of linter grade cellulose (approximately 15 mm×15 mm in size; available from Buckeye Technologies, Inc.) were pre-dried at 55° C. in a vacuum oven, <25 inch Hg, for no less than 24 hours and stored in a 0% humidity dessicator until use. The PTFE stopper was removed and dried hand-torn chips of linter grade cellulose (15.16 g, 0.0935 moles) were added as quickly as possible to the reaction mixture via a wide-mouth powder funnel—it was ensured that there was a minimal positive argon pressure flowing through the system when performing this operation in order to minimize vapor release from the reaction vessel. After complete addition of cellulose, the stopper was replaced; and the reaction mixture was heated incrementally, using a temperature-controlled oil bath, over the next 2 hours to achieve a final internal solution temperature of 50° C. This mixture was held under these conditions with constant stirring for 18 hours. Heating of the mixture was ceased after this period.

Neutralization—After cooling the constantly stirred dope to an internal temperature of 30° C., 35 mL of trichloroethylene (Aldrich; Catalog No. 372145; anhydrous) was added in order to both decrease the viscosity of the dope and also to aid in the dissolution of the neutralization cocktail. The residual trifluoroacetic anhydride was neutralized via slow, drop-wise addition of a 50:50 (w/w) mixture (molar excess of 10% in relation to excess TFAA) of methanol/Millipore water (4.18 g and/or 5.28 mL MeOH & 2.35 g H₂O) over a period of 90 minutes. In this case, the internal temperature rose to a maximum of 42.5° C. (12.5° C. increase). The rate of addition was maintained such as to maintain the internal temperature at or below 50° C. to minimize possible degradation of cellulose. After the internal temperature returned to 30-35° C., the neutralization was determined to be essentially complete. The reaction vessel was then prepared for distillation.

Distillation—Before distillation, both Claisen adapters, the thermocouple/thermometer adapter, the addition funnel and condenser were removed and replaced with glass stoppers lubricated with DOW Corning High Vacuum Grease. A 90° hose connection adapter was added to open neck of the 4-Neck, 1000 mL round-bottom flask and was connected in series, via high vacuum hose and Schlenk manifold, to two 500 mL vacuum traps. Both traps were cryogenically cooled with liquid nitrogen. While maintaining the internal temperature of the mixture at 30° C., vacuum was slowly/incrementally introduced until reaching an ultimate vacuum of 60 mmHg. After equilibrating at this pressure, distillation of residual trifluoroacetic acid, trifluoroacetic acid methyl ester, water and methanol was initiated by heating the crude mixture—the heat was slowly ramped as to induce/maintain a steady distillation. Heating was continued until the internal temperature reached a maximum of 50° C. With constant agitation, full vacuum was maintained at 50° C. for a period of 60 minutes. At this time, the distillation of the vast majority of lower boiling contaminants and a great deal of trichloroethylene were removed. Heating was ceased, vacuum terminated, and the system was backfilled with argon. The resultant mixture was a very viscous dope that had manifested a darker, brown-red color.

Precipitation/Isolation—In order to prepare for precipitation, 50 mL of trichloroethylene (Aldrich, Catalog No. 372145; anhydrous) was added and stirred into the resultant dope for a period of 30 minutes—trichloroethylene not only acts as a thinning agent in regards to the solution viscosity but also aids in the efficient removal of residual/un-reacted fatty acid. The dope was transferred from the parent reactor to a separatory funnel of an appropriate size. The separatory funnel was then used to slowly meter the crude dope into a vigorously stirred/highly agitated solution of 100% methanol (Burdick & Jackson; Catalog No. 230-4)—the ratio for the total amount of methanol required to effectively precipitate the dope was on the order of 20-25:1 [(v/v); MeOH/dope]. After precipitation was complete, the off-white, fine precipitate was left to stir slowly in the precipitation vessel for an additional 60-120 minutes in order to harden the material. Upon termination of stirring, all of the material, in the form of very fine, sand-like particles, instantaneously fell to the bottom of the precipitation vessel and coagulated into an even, thin patty, suggesting that the purified material was very dense in nature. The precipitate was captured via vacuum filtration through a course, glass-fritted funnel. The solid was immediately transferred to an appropriately-sized flask and slurried with 2000 mL of methanol. The resultant slurry was stirred for an additional hour and left to settle and further harden for a period of no less than 12 hours. The isolated polymer was captured via vacuum filtration through a course, glass-fritted funnel; left in the funnel with vacuum still applied to air-dry for at least 60 minutes; crushed to a grainy particle size in a mortar and pestle; and transferred to a drying dish in preparation for further solvent and water removal. The air-dried material was further dried in a vacuum oven, <25 inch Hg, at 50° C. for no less than 24 hours. The material was removed from the vacuum oven and transferred to a dessicator in order for the polymer to cool prior to obtaining samples for analytical analysis and/or solubility testing. To complete processing, the particle size of the dried material was reduced via passage through a small-scale Hammermill fitted with a 1.0 mm screen filter. The reaction to produce cellulose neo-decanoate (and/or cellulose tri-O-neo-decanoate) yielded 49.35 g (84.4% yield, isolated) of a finely powdered, grainy solid that was dull white in color. NMR procedures and related calculations were conducted using the procedures described above in Example 39, to minimize the possibility that NMR results do not include moles residual reactants as part of the moles of ester. The material was found by ¹H NMR to possess a neo-decanoate DS of 2.53; the polymer was soluble in both isohexadecane and isododecane. The protocol for solubility determination set forth above in Comparative Example 1 was conducted in isohexadecane and isododecane at the concentrations in the tables set forth below. Results were recorded after a period of 2 up to one month. Please see adjoining tables (below) for detailed analytical and solubility results.

### Example 41

### Synthesis and Purification of Cellulose Neo-Decanoate

Preparation of the mixed neo-decanoic acid/tri-fluoroacetic acid anhydride To a flame dried, argon-purged 1000 mL 4-Neck round-bottom flask; equipped with a stirrer bearing/mechanical stir shaft designed for vacuum applications, a PTFE stopper (24/40 standard taper joint) with extracting nut, and two Claisen adapters that were fitted with the following: a) reflux condenser b) 125 mL addition funnel with metering stopcock c) internal thermometer/thermocouple; was added 100.97 g of Neo-Decanoic Acid Prime, CAS# 26896-20-8, available from ExxonMobil Chemical Company, a neo-decanoic acid product (clear, colorless liquid at room temperature/pressure; ¹H NMR, ¹³C NMR, and Liquid Chromatography (LC) provide evidence for a multi-component mixture consisting of greater than 6 compounds). In order to minimize color of the mixed anhydride, the solution was cooled to 0° C. and held for 60 minutes. Trifluoroacetic anhydride (TFAA) (111.92 g, 0.5329 moles; 74.07 mL) (Aldrich; Catalog No. 106232) was quickly added to a 125 mL addition funnel equipped with a fine-control metering stopcock. The glass stopper was returned to the addition funnel, and it was ensured that the positive pressure flowing through the reactor was at the lowest level possible (slowest possible bubble rate in an oil bubbler filled with silicone oil). TFAA was added to the cooled solution at a moderate rate over the course of between 120-150 minutes (2.0-2.5 hours)—the rate of addition was such as to maintain the internal temperature of the reaction mixture below 5° C. During TFAA addition, the solution began to take on a pinkish purple to reddish purple hue. After complete addition of trifluoroacetic anhydride, the cooling bath was removed and replaced with a bath containing tap water at room temperature. After 25-30 minutes (or when the internal temperature of the flask returned to 25° C.), the mixture was stirred for an additional 60 minutes at room temperature to ensure that the mixed anhydride was formed. At this point, the reaction mixture was homogeneous and had developed a very brilliant reddish purple hue.

Reaction of cellulose with the mixed anhydride of neo-decanoic acid and tri-fluoroacetic acid—Torn chips of linter grade cellulose (approximately 15 mm×15 mm in size; available from Buckeye Technologies, Inc.) were pre-dried at 55° C. in a vacuum oven, <25 inch Hg, for no less than 24 hours and stored in a 0% humidity dessicator until use. The PTFE stopper was removed and dried hand-torn chips of linter grade cellulose (15.16 g, 0.0935 moles) were added as quickly as possible to the reaction mixture via a wide-mouth powder funnel—it was ensured that there was a minimal positive argon pressure flowing through the system when performing this operation in order to minimize vapor release from the reaction vessel. After complete addition of cellulose, the stopper was returned; and the reaction mixture was heated incrementally, using a temperature-controlled oil bath, over the next 5 hours to achieve a final internal solution temperature of 53.5° C. This mixture was held under these conditions with constant stirring for 29 hours. At this time the mixture had reacted to yield a semi-translucent, gritty dope that was a dark, burgundy hue, containing a small amount of light-brown highlight in the overall color. In an attempt to drive the reaction equilibrium towards tri-ester, 40 mL of trichloroethylene (Aldrich; Catalog No. 372145) was added; the temperature of the oil-bath was decreased to 45° C.; and the reaction was continued for an additional 9.5 hours. However, by the physical appearance and consistency of the dope, it was apparent that the degree of substitution for the ester was most likely lower than that obtained for the like polymer produced in Example 40. Even so, heating of the mixture was ceased after this period-total reaction time of 38.5 hours.

Neutralization—After cooling, the residual trifluoroacetic anhydride was neutralized via slow, drop-wise addition of a 50:50 (w/w) mixture (molar excess of 10% in relation to excess TFAA) of methanol/Millipore water (4.18 g and/or 5.28 mL MeOH & 2.35 g H₂O) over a period of 90 minutes. In this case, the internal temperature rose to a maximum of 42.5° C. (12.5° C. increase). The rate of addition was maintained such as to maintain the internal temperature at or below 50° C. to minimize possible degradation of cellulose. After the internal temperature returned to 30-35° C., the neutralization was determined to be essentially complete. The reaction vessel was then prepared for distillation.

Distillation—Before distillation, both Claisen adapters, the thermocouple/thermometer adapter, the addition funnel and condenser were removed and replaced with glass stoppers lubricated with DOW Corning High Vacuum Grease. A 90° hose connection adapter was added to open neck of the 4-Neck, 1000 mL round-bottom flask and was connected in series, via high vacuum hose and Schlenk manifold, to two 500 mL vacuum traps. Both traps were cryogenically cooled with liquid nitrogen. While maintaining the internal temperature of the mixture at 30° C., vacuum was slowly/incrementally introduced until reaching an ultimate vacuum of 60 mmHg. After equilibrating at this pressure, distillation of residual trifluoroacetic acid, trifluoroacetic acid methyl ester, water and methanol was initiated by heating the crude mixture—the heat was slowly ramped as to induce/maintain a steady distillation. Heating was continued until the internal temperature reached a maximum of 50° C. With constant agitation, full vacuum was maintained at 50° C. for a period of 60 minutes. At this time, the distillation of the vast majority of lower boiling contaminants and a great deal of trichloroethylene were removed. Heating was ceased, vacuum terminated, and the system was backfilled with argon. The resultant mixture was a very viscous, gritty dope that had manifested a darker, brown-red color.

Precipitation/Isolation—In order to prepare for precipitation, 60 mL of trichloroethylene (Aldrich; Catalog No. 372145) was added and stirred into the resultant dope for a period of 30 minutes. The dope was transferred from the parent reactor to a separatory funnel of an appropriate size. The separatory funnel was then used to slowly meter the crude dope into a vigorously stirred/highly agitated solution of 100% methanol (Burdick & Jackson; Catalog No. 230-4)—the ratio for the total amount of methanol required to effectively precipitate the dope was on the order of 20-25:1 [(v/v); MeOH/dope]. After precipitation was complete, the off-white, fine precipitate was left to stir slowly in the precipitation vessel for an additional 60-120 minutes in order to harden the material. Upon termination of stirring, all of the material, in the form of very fine, sand-like particles, instantaneously fell to the bottom of the precipitation vessel and coagulated into an even, thin patty, suggesting that the purified material was very dense in nature. The precipitate was captured via vacuum filtration through a course, glass-fritted funnel. The solid was immediately transferred to an appropriately-sized flask and slurried with 2000 mL of methanol. The resultant slurry was stirred for an additional hour and left to settle and further harden for a period of no less than 12 hours. The isolated polymer was captured via vacuum filtration through a course, glass-fritted funnel; left in the funnel with vacuum still applied to air-dry for at least 60 minutes; crushed to a grainy particle size in a mortar and pestle; and transferred to a drying dish in preparation for further solvent and water removal. The air-dried material was further dried in a vacuum oven, <25 inch Hg, at 50° C. for no less than 24 hours. The material was removed from the vacuum oven and transferred to a dessicator in order for the polymer to cool prior to obtaining samples for analytical analysis and/or solubility testing. To complete processing, the particle size of the dried material was reduced via passage through a small-scale Hammermill fitted with a 1.0 mm screen filter. The reaction to produce cellulose neo-decanoate (and/or cellulose tri-O-neo-decanoate) yielded 41.97 g (71.8% yield, isolated) of a finely powdered, grainy solid that was dull white in color. NMR procedures and related calculations were conducted using the procedures described above in Example 39, to minimize the possibility that NMR results do not include moles residual reactants as part of the moles of ester. The material was found by ¹H NMR to possess a neo-decanoate DS of 2.35. The protocol for solubility determination set forth above in Comparative Example 1 was conducted in isohexadecane and isododecane at the concentrations in the tables set forth below. Results were recorded after a period of 2 up to one month. Please see adjoining tables (below) for detailed analytical and solubility results. In stark contrast to the DS 2.53 material prepared from the same fatty acid product in Example 40, the polymer had very poor solubility in both isohexadecane and isododecane.

### Example 42

### Synthesis and Purification of Cellulose Neo-Heptanoate

Preparation of the mixed neo-heptanoic acid/tri-fluoroacetic acid anhydride—To a flame dried, argon-purged 1000 mL 4-Neck round-bottom flask; equipped with a stirrer bearing/mechanical stir shaft designed for vacuum applications, a PTFE stopper (24/40 standard taper joint) with extracting nut, and two Claisen adapters that were fitted with the following: a) reflux condenser b) 125 mL addition funnel with metering stopcock c) internal thermometer/thermocouple; was added 76.31 g of Neo-Heptanoic Acid Prime, CAS# 95823-36-2; a neo-heptanoic acid product available from ExxonMobil Chemical Company (clear, colorless liquid at room temperature/pressure; ¹H NMR, ¹³C NMR, and Liquid Chromatography (LC) provide evidence for a multi-component mixture with at least 3-4 differing compounds). In order to minimize color of the mixed anhydride, the solution was cooled to 0° C. and held for 60 minutes. Trifluoroacetic anhydride (TFAA) (111.92 g, 0.5329 moles; 74.07 mL) (Aldrich; Catalog No. 106232) was quickly added to a 125 mL addition funnel equipped with a fine-control metering stopcock. The glass stopper was returned to the addition funnel, and it was ensured that the positive pressure flowing through the reactor was at the lowest level possible (slowest possible bubble rate in an oil bubbler filled with silicone oil). TFAA was added to the cooled solution at a moderate rate over the course of between 120-150 minutes (2.0-2.5 hours)—the rate of addition was such as to maintain the internal temperature of the reaction mixture below 5° C. During TFAA addition, the solution began to take on a slight light pink hue. After complete addition of trifluoroacetic anhydride, the cooling bath was removed and replaced with a bath containing tap water at room temperature. After 25-30 minutes (or when the internal temperature of the flask returned to 25° C.), the mixture was stirred for an additional 60 minutes at room temperature to ensure that the mixed anhydride was formed. At this point, the reaction mixture was homogeneous and had developed a prevalent light pinkish rose hue.

Reaction of cellulose with the mixed anhydride of neo-heptanoic acid and tri-fluoroacetic acid—Torn chips of linter grade cellulose (approximately 115 mm×15 mm in size; available from Buckeye Technologies, Inc.) were pre-dried at 55° C. in a vacuum oven, <25 inch Hg, for no less than 24 hours and stored in a 0% humidity dessicator until use. The PTFE stopper was removed and dried hand-torn chips of linter grade cellulose (15.17 g, 0.0936 moles) were added as quickly as possible to the reaction mixture via a wide-mouth powder funnel—it was ensured that there was a minimal positive argon pressure flowing through the system when performing this operation in order to minimize vapor release from the reaction vessel. After complete addition of cellulose, the stopper was returned; and the reaction mixture was heated incrementally, using a temperature-controlled oil bath, over the next 5 hours to achieve a final internal solution temperature of 53.5° C. At this point, the stirred mixture was a creamy-to-almond color and looked very similar to carnauba wax in regards to its consistency. This mixture was held under these conditions with constant stirring for 20 hours. At this time the mixture had reacted to yield a translucent dope that was a light, golden brown hue. The oil bath temperature was decreased as to achieve an internal temperature of 30° C. for the dope. In order to thin the viscous mixture in preparation for neutralization, 60 mL of trichloroethylene (Aldrich; Catalog No. 372145) was added.

Neutralization—After cooling, the residual trifluoroacetic anhydride was neutralized via slow, drop-wise addition of a 50:50 (w/w) mixture (molar excess of 10% in relation to excess TFAA) of methanol/Millipore water (4.18 g and/or 5.28 mL MeOH & 2.35 g H₂O) over a period of 90 minutes. In this case, the internal temperature rose to a maximum of 41.5° C. (11.5° C. increase). The rate of addition was such as to maintain the internal temperature at or below 50° C. to minimize possible degradation of cellulose. After the internal temperature returned to 30-35° C., the neutralization was determined to be essentially complete. The reaction vessel was then prepared for distillation.

Distillation—Before distillation, both Claisen adapters, the thermocouple/thermometer adapter, the addition funnel and condenser were removed and replaced with glass stoppers lubricated with DOW Corning High Vacuum Grease. A 90° hose connection adapter was added to open neck of the 4-Neck, 1000 mL round-bottom flask and was connected in series, via high vacuum hose and Schlenk manifold, to two 500 mL vacuum traps. Both traps were cryogenically cooled with liquid nitrogen. While maintaining the internal temperature of the mixture at 30° C., vacuum was slowly/incrementally introduced until reaching an ultimate vacuum of 60 mmHg. After equilibrating at this pressure, distillation of residual trifluoroacetic acid, trifluoroacetic acid methyl ester, water and methanol was initiated by heating the crude mixture—the heat was slowly ramped as to induce/maintain a steady distillation. Heating was continued until the internal temperature reached a maximum of 45° C. With constant agitation, full vacuum was maintained at 45° C. for a period of 60 minutes. At this time, the distillation of the vast majority of lower boiling contaminants and a great deal of trichloroethylene were removed. Heating was ceased, vacuum terminated, and the system was backfilled with argon. The resultant mixture was a very viscous dope that had manifested a darker, brownish gold color.

Precipitation/Isolation—In order to prepare for precipitation, 60 mL of trichloroethylene (Aldrich; Catalog No. 372145) was added and stirred into the resultant dope for a period of 30 minutes—trichloroethylene not only acts as a thinning agent in regards to the solution viscosity but also aids in the efficient removal of residual/un-reacted fatty acid. The dope was transferred from the parent reactor to a separatory funnel of an appropriate size. The separatory funnel was then used to slowly meter the crude dope into a vigorously stirred/highly agitated solution of 100% methanol (Burdick & Jackson; Catalog No. 230-4)—the ratio for the total amount of methanol required to effectively precipitate the dope was on the order of 20-25:1 [(v/v); MeOH/dope]. After precipitation was complete, the white, powdery precipitate was left to stir slowly in the precipitation vessel for an additional 60-120 minutes in order to harden the material. Upon termination of stirring, all of the material, in the form of very fluffy, swollen white particles began to slowly cascade to the bottom of the precipitation vessel and coagulated into a thick, flocculating precipitate. The precipitate was captured via vacuum filtration through a course, glass-fritted funnel. The solid was immediately transferred to an appropriately-sized flask and slurried with 2000 mL of methanol. The resultant slurry was stirred for an additional hour and left to settle and further harden for a period of no less than 12 hours. Even after standing for 12 hours, approximately 5-10% of the precipitate remained freely suspended above the main mass of polymer that had fallen to the bottom of the precipitation vessel. The isolated polymer was captured via vacuum filtration through a course, glass-fritted funnel and left in the funnel with vacuum still applied to air-dry for at least 60 minutes. The air-dried material was further dried in a vacuum oven, <25 inch Hg, at 50° C. for no less than 24 hours. The material was removed from the vacuum oven and transferred to a dessicator in order for the polymer to cool prior to obtaining samples for analytical analysis and/or solubility testing. The reaction to produce cellulose neo-heptanoate (and/or cellulose tri-O-neo-heptanoate) yielded 44.77 g (95.9% yield, isolated) of a fluffy-to-feathery solid that was bright white in color. NMR procedures and related calculations were conducted using the procedures described above in Example 39, to minimize the possibility that NMR results do not include moles residual reactants as part of the moles of ester. The material was found by ¹H NMR to possess a neo-heptanoate DS of 2.85; the polymer was soluble in both isohexadecane and isododecane. The protocol for solubility determination set forth above in Comparative Example 1 was conducted in isohexadecane and isododecane at the concentrations in the tables set forth below. Results were recorded after a period of 2 up to one month. Please see adjoining tables (below) for detailed analytical and solubility results.

### Example 43

### Synthesis and Purification of Cellulose (2-Ethylhexanoate)

Preparation of the mixed 2-ethylhexanoic acid/tri-fluoroacetic acid anhydride. 84.53 g of 2-ethylhexanoic acid (Aldrich Catalog No. 538701, manufactured by Eastman Chemical Company) were added to a flame dried, argon-purged 1000 mL 4-neck round-bottom flask equipped with a stirrer bearing/mechanical stir shaft designed for vacuum applications, a PTFE stopper (24/40 standard taper joint) with extracting nut, and two Claisen adapters that were fitted with the following: a) reflux condenser; b) 125 mL addition funnel with metering stopcock; and c) internal thermometer/thermocouple. All ground glass joints were fitted with PTFE sleeves to avoid reaction of grease with trifluoroacetic anhydride. The exhaust port of the reaction vessel, coupled in series with a Claisen adapter, was connected via tubing to a 500 mL Dreschel gas-washing bottle that contained 350 mL of a saturated, aqueous solution of sodium bicarbonate (NaHCO₃). The wash bottle was utilized to scrub trifluoroacetic acid that escapes during reaction. The solution was cooled to 0° C. and held for 60 minutes. 111.92 g (74.07 mL) of trifluoroacetic anhydride (TFAA) (Aldrich; Catalog # 106232; used as received) was then quickly added to a 125 mL addition funnel equipped with a fine-control metering stopcock. The glass stopper was returned to the addition funnel and care was then taken to assure that positive pressure flowing through the reactor was maintained low by attempting to obtain slowest possible bubble rate in an oil bubbler filled with silicone oil. TFAA was added to the cooled solution at a moderate rate over the course of between 120-150 minutes (2.0-2.5 hours) while the internal temperature of the reaction mixture was maintained below 5° C. After complete addition of trifluoroacetic anhydride, the cooling bath was removed and replaced with a bath containing tap water at room temperature. When the internal temperature of the flask returned to 25° C., the mixture was stirred for an additional 60 minutes at room temperature to ensure that the mixed anhydride was formed.

Reaction of cellulose with the mixed anhydride of 2-ethylhexanoic acid and tri-fluoroacetic acid: Prior to reaction, torn chips (approximately 15 mm×15 mm in size) of linter grade cellulose (available from Buckeye Technologies, Inc.) were pre-dried at 55° C. in a vacuum oven, <25 inch Hg, for no less than 24 hours. then quickly transferred to a 0% humidity dessicator and stored until use. The PTFE stopper was removed and 15.43 g of the torn chips were quickly added to the reaction mixture via a wide-mouth powder funnel while maintaining minimal positive argon pressure flowing through the system. After complete addition of cellulose, the stopper was returned and the reaction mixture was heated incrementally, using a temperature-controlled oil bath, over the next 2.5 hours to achieve a final internal solution temperature of 50.0° C. Only one very small piece of cellulose was visibly not in solution. This mixture was held under these conditions with constant stirring for 6 hours. The oil bath temperature was decreased as to achieve an internal temperature of 35° C. 35 mL of trichloroethylene (Aldrich; Catalog # 372145, used as received) was then added.

Neutralization—After cooling, the residual trifluoroacetic anhydride was neutralized via slow, drop-wise addition of a mixture of methanol/Millipore water (4.18 g MeOH & 2.35 g H₂O) (molar excess of 10% in relation to excess TFAA) over a period of 50 minutes. The rate of addition was controlled to maintain the internal temperature at or below 50° C. and the internal temperature rose to a maximum of 40.5° C. After the internal temperature returned to 30-35° C., the reaction vessel was then prepared for distillation.

Distillation—Before distillation, both Claisen adapters, the thermocouple/thermometer adapter, the addition funnel and condenser were removed and replaced with glass stoppers lubricated with a high vacuum grease. A 90° hose connection adapter was added to the open neck of the 4-Neck, 1000 mL round-bottom flask and was connected in series, via high vacuum hose and Schlenk manifold, to two 500 mL vacuum traps. Both traps were cryogenically cooled with liquid nitrogen while maintaining the internal temperature of the mixture at 30° C. Vacuum was slowly/incrementally introduced until reaching an ultimate vacuum of 58-61 mmHg. After equilibrating at this pressure, distillation of residual trifluoroacetic acid, trifluoroacetic acid methyl ester, water and methanol was initiated by heating the crude mixture. The heat was slowly increased to induce/maintain a steady distillation. Heating continued until the internal temperature reached a maximum of 45.6° C. With constant agitation, full vacuum (54 mm Hg) was maintained at 45.6° C. for a period of 60 minutes. Heating was ceased, vacuum terminated, and the system was backfilled with argon. The resultant mixture was a very viscous dope that had manifested a darker, brownish gold color.

Precipitation/Isolation—40 mL of trichloroethylene (Aldrich; Catalog # 372145; used as received) was added and stirred into the resultant dope for a period of 30 minutes to reduce viscosity and to aid in the efficient removal of residual/un-reacted fatty acid. The dope was transferred from the parent reactor to a separatory funnel of an appropriate size. The separatory funnel was then used to slowly meter the crude dope into a vigorously stirred/highly agitated solution of 100% methanol (Burdick & Jackson; Cat. # 230-4; 99.9+% by GC). The ratio for the total amount of methanol required to effectively precipitate the dope was on the order of 20-25:1 [(v/v); MeOH/dope]. After precipitation was complete, the white, powdery precipitate was left to stir slowly in the precipitation vessel for an additional 60-120 minutes in order to harden the material. Upon termination of stirring, all of the material, in the form of very fluffy, swollen white particles began to slowly cascade to the bottom of the precipitation vessel and coagulated into a thick, flocculating precipitate. The precipitate was captured via vacuum filtration through a course, glass-fritted funnel. The solid was immediately transferred to an appropriately-sized flask and slurried with 2000 mL of methanol. The resultant slurry was stirred for an additional hour and left to settle and further harden for a period of no less than 12 hours. The isolated polymer was captured via vacuum filtration through a glass-fritted funnel and left in the funnel with vacuum still applied to air-dry for at least 60 minutes. The air-dried material was further dried in a vacuum oven, <25 inch Hg, at 50° C. for no less than 24 hours. The material was removed from the vacuum oven and transferred to a dessicator prior to obtaining samples for analytical analysis and/or solubility testing. The reaction yielded 48.36 g (95.6% yield, isolated) of a fluffy-to-feathery solid that was dull-to-off white in color. The material was found by ¹H NMR to possess a 2-ethylhexanoate DS of 2.93; the polymer was very highly soluble in both isohexadecane and isododecane. Detailed analytical and solubility results are presented in Tables 14, 15 and 20.

TABLE 14

| | Cellulose | | | Residual | Residual | Residual |
|---|---|---|---|---|---|---|
| | Ester | | DS | Fluoride^{b} | TFA^{b} | Fatty |
| Example | (Branch | DS | Free- | (ppm- | (ppm- | Acid^{c} |
| Number | Type) | ester^{a} | OH^{a} | wt) | wt) | (wt %) |
| 39 | stearate | 2.81 | n.o.^{d} | <5 | 437 | <0.07 |
| 40 | neo- | 2.53 | 0.28 | <5 | 343 | <0.05 |
| | decanoate | | | | | |
| 41 | neo- | 2.35 | 0.26 | n.a.^{e} | n.a.^{e} | n.a.^{e} |
| | decanoate | | | | | |
| 42 | neo- | 2.85 | 0.05 | <5 | 67.3 | <0.04 |
| | heptanoate | | | | | |
| 43 | 2-ethyl | 2.93 | n.o.^{d} | <5 | 181 | <0.04 |
| | hexanoate | | | | | |

^{a}Degree of Substitution (DS) determined by for both the ester moiety and free-Hydroxyl (Free-OH).
^{b}Residual Fluoride and Trifluoroacetic Acid (TFA) determined by Ion Chromatography (IC).
^{c}Residual Fatty Acid (i.e. unreacted stearic, neo-decanoic, and neo-heptanoic acids) determined by Liquid Chromatography (LC).
^{d}None observed (n.o.).
^{e}Not available (n.a.)-Residual analysis was unable to be obtained due to the inability to solvate/dissolve this polymer in the necessary solvent systems.

TABLE 15

| | Cellulose | | | |
|---|---|---|---|---|
| | Ester | Decomposition | Relaxation^{b} | Melting^{b} |
| Example | (Branch | Onset^{a} | T_{g} | Tₘ |
| Number | Type) | (° C.) | (° C.) | (° C.) |
| 39 | stearate | 358 | n.o.^{c} | 26.8 (primary; v. |
| | | | | broad) |
| | | | | 105.6 |
| | | | | (secondary; |
| | | | | broad) |
| 40 | neo- | 333 | 33.7 | n.o.^{c} |
| | decanoate | | | |
| 41 | neo- | 333 | 38.7 | n.o.^{c} |
| | decanoate | | | |
| 42 | neo- | 359 | 97.6 | n.o.^{c} |
| | heptanoate | | | |
| 43 | 2-ethyl | 358 | 31.9 | n.o.^{c} |
| | hexanoate | | | |

^{a}Determined by Thermogravimetric Analysis (TGA) and recorded as the value required to achieve 10% total mass loss under nitrogen at a scan rate of 20° C./minute.
^{b}Determined by Differential Scanning Calorimetry (DSC). Reported values for relaxation events/glass transitions (Tg) and melting transitions (Tm) were obtained during the second heating at a scan rate of 20° C./minute.
^{c}None observed (n.o.) - Specified thermal event was not observed in the range of −140 to 175° C.

In the following tables, “Soluble” denotes formation of a liquid, a liquid with small gels, or a gel, with or without expunged solvent (in all cases irrespective of clarity or haziness); whereas, “Not soluble” means phase separation occurs or solid particles of cellulose ester compound remain.

TABLE 16
Solubility Properties of Cellulose Stearate (Example 39) in Hydrocarbon
Solvents of Interest

Solubility Properties
in the Specified Solvent

| % Concentration | Isohexadecane | Isododecane |
|---|---|---|
| (w/w)^{a} | (IHD) | (IDD) |
| 1 | Soluble | Soluble |
| 2 | Soluble | Soluble |
| 5 | Soluble | Soluble |
| 8 | Soluble | Soluble |
| 10 | Soluble | Soluble |
| 15 | Soluble | Soluble |
| 20 | Soluble | Soluble |

^{a}weight of cellulose ester compound over total weight of solution.

TABLE 17
Solubility Properties of Cellulose Neo-Decanoate (Example 40)
in Hydrocarbon Solvents of Interest

Solubility Properties
in the Specified Solvent

| % Concentration | Isohexadecane | Isododecane |
|---|---|---|
| (w/w)^{a} | (IHD) | (IDD) |
| 1 | Soluble | Soluble |
| 2 | Soluble | Soluble |
| 5 | Soluble | Soluble |
| 8 | Soluble | Soluble |
| 10 | Soluble | Soluble |
| 15 | Soluble | Soluble |
| 20 | Soluble | Soluble |
| 30 | Soluble | Soluble |
| 40 | Soluble | Soluble |
| 50 | Soluble | Soluble |

^{a}weight of cellulose ester compound over total weight of solution.

TABLE 18
Solubility Properties of Cellulose Neo-Decanoate (Example 41)
in Hydrocarbon Solvents of Interest

Solubility Properties
in the Specified Solvent

| % Concentration | Isohexadecane | Isododecane |
|---|---|---|
| (w/w)^{a} | (IHD) | (IDD) |
| 1 | Insoluble | Insoluble |
| 2 | Insoluble | Insoluble |
| 5 | Insoluble | Insoluble |
| 8 | Insoluble | Insoluble |
| 10 | Insoluble | Insoluble |
| 15 | Insoluble | Insoluble |
| 20 | Insoluble | Insoluble |
| 30 | Soluble?* | Soluble?* |
| 40 | Insoluble | Soluble?* |
| 50 | Insoluble | Insoluble |

^{a}weight of cellulose ester compound over total weight of solution.
*Met criteria for definition of “Soluble” above but very hazy liquid or gel with physical inconsistency; unclear whether dissolution actually occurs.

TABLE 19
Solubility Properties of Cellulose Neo-Heptanoate (Example 42) in
Hydrocarbon Solvents of Interest

Solubility Properties
in the Specified Solvent

| % Concentration | Isohexadecane | Isododecane |
|---|---|---|
| (w/w)^{a} | (IHD) | (IDD) |
| 1 | Soluble | Soluble |
| 2 | Soluble | Soluble |
| 5 | Soluble | Soluble |
| 8 | Soluble | Soluble |
| 10 | Soluble | Soluble |
| 15 | Soluble | Soluble |
| 20 | Soluble | Soluble |
| 30 | Soluble | Soluble |
| 40 | Soluble | Soluble |
| 50 | Soluble | Soluble |

^{a}weight of cellulose ester compound over total weight of solution.

TABLE 20
Solubility Properties of Cellulose 2-Ethylhexanoate (Example 43) in
Hydrocarbon Solvents of Interest

Solubility Properties
in the Specified Solvent

| % Concentration | Isohexadecane | Isododecane |
|---|---|---|
| (w/w)^{a} | (IHD) | (IDD) |
| 1 | Soluble | Soluble |
| 2 | Soluble | Soluble |
| 5 | Soluble | Soluble |
| 8 | Soluble | Soluble |
| 10 | Soluble | Soluble |
| 15 | Soluble | Soluble |
| 20 | Soluble | Soluble |
| 30 | Soluble | Soluble |
| 40 | Soluble | Soluble |
| 50 | Soluble | Soluble |

^{a}weight of cellulose ester compound over total weight of solution.

Having described the invention in detail, those skilled in the art will appreciate that modifications may be made to the various aspects of the invention without departing from the scope and spirit of the invention disclosed and described herein. It is, therefore, not intended that the scope of the invention be limited to the specific embodiments illustrated and described but rather it is intended that the scope of the present invention be determined by the appended claims and their equivalents.

We claim:

1. A composition comprising:
at least one cellulose compound upon which at least some free hydroxyl groups are substituted by at least one ester moiety having at least 6 carbon atoms and
at least one lipophilic solvent;
wherein the total degree of substitution for the ester moieties on the at least one cellulose compound is at least about 2.5 and wherein at least some of the at least one cellulose compound is dissolved in the at least one lipophilic solvent.

2. The composition of claim 1, wherein the at least one ester moiety is selected from saturated branched ester moieties, saturated unbranched ester moieties, branched and unsaturated ester moieties and combinations of two or more of the foregoing.

3. The composition of claim 1, wherein the at least one ester moiety is selected from saturated and unsaturated branched ester moieties and combinations of two or more of the foregoing.

4. The composition of claim 1, wherein the at least one ester moiety is selected from ester moieties having a tert-butyl terminus, esters having a saturated iso-alkyl group, esters having an alkyl structure that is branched at the alpha carbon, and combinations of two or more of the foregoing.

5. The composition of claim 1, wherein the at least one ester moiety is selected from unsaturated ester moieties and combinations of two or more of the foregoing.

6. The composition of claim 1, wherein the at least one cellulose compound dissolves in isododecane at concentrations of at least about 5% by weight at room temperature and standard pressure.

7. The composition of claim 1, wherein the at least one cellulose compound dissolves in isododecane at concentrations of at least about 30% by weight at room temperature and standard pressure.

8. The composition of claim 1, wherein the composition is a deodorant, an antiperspirant, a shaving product, a skin lotion, a moisturizing product, a skin toner, a cleansing product, a hair care product, a manicure product, a protective cream, a sunscreen product, an insect repellent, an anti-aging product, a color cosmetic, a lipstick, a cosmetic foundation, a face powder, an eye liner, an eye shadow, a blush, a mascara, a system for topical application of medicaments to the skin, or a combination of two or more of the foregoing.

9. The composition of claim 1, wherein the cellulose compound is derived from wood pulp or cotton linters.

10. A composition comprising:
at least one cellulose compound upon which at least some free hydroxyl groups are substituted by at least one first ester moiety having at least 6 carbon atoms at least some additional free hydroxyl groups are substituted by at least one second ester moiety and
at least one lipophilic solvent;
wherein the total degree of substitution for the first ester moiety on the at least one cellulose compound is at least about 0.8 and wherein at least some of the at least one cellulose compound is dissolved in the at least one lipophilic solvent.

11. The composition of claim 10, wherein the total degree of substitution for the first ester moiety on the at least one cellulose compound is at least about 1.0.

12. The composition of claim 10, wherein the total degree of substitution for the at least one first ester moiety on the at least one cellulose compound is at least about 1.5.

13. The composition of claim 10, wherein the at least first one ester moiety is selected from saturated branched ester moieties, saturated unbranched moieties, branched and unsaturated moieties and combinations of two or more of the foregoing.

14. The composition of claim 10, wherein the at least one first ester moiety is selected from saturated and unsaturated branched ester moieties and combinations of two or more of the foregoing.

15. The composition of claim 10, wherein the at least one first ester moiety is selected from ester moieties having a tert-butyl terminus, esters having a saturated iso-alkyl group, esters having an alkyl structure that is branched at the alpha carbon, and combinations of two or more of the foregoing.

16. The composition of claim 10, wherein the at least one first ester moiety is selected from unsaturated ester moieties and combinations of two or more of the foregoing.

17. The composition of claim 10, wherein the at least one second ester moiety is selected from acetate, propionate, butyrates, pentanoates, and combinations of two or more of the foregoing.

18. The composition of claim 10, wherein the composition is a deodorant, an antiperspirant, a shaving product, a skin lotion, a moisturizing product, a skin toner, a cleansing product, a hair care product, a manicure product, a protective cream, a sunscreen product, an insect repellent, an anti-aging product, a color cosmetic, a lipstick, a cosmetic foundation, a face powder, an eye liner, an eye shadow, a blush, a mascara, a system for topical application of medicaments to the skin, or a combination of two or more of the foregoing.

19. A composition comprising:
at least one cellulose compound upon which at least some free hydroxyl groups are substituted by at least one ester moiety having at least 6 carbon atoms and selected from saturated branched ester moieties, saturated unbranched ester moieties, branched and unsaturated ester moieties and combinations of two or more of the foregoing, and
at least one lipophilic solvent;
wherein at least some of the at least one cellulose compound is dissolved in the at least one lipophilic solvent.
